(19) United States
(12) Patent Application Publication
(10) Pub. No.: US 2012/0157377 A1
(43) Pub. Date: Jun. 21, 2012

(54) METHODS TO ENHANCE NIGHT VISION AND TREATMENT OF NIGHT BLINDNESS

(76) Inventor: Totada R. Shantha, Stone Mountain, GA (US)

(21) Appl. No.: 13/409,076

(22) Filed: Feb. 29, 2012

(51) Int. Cl. A61K 38/28; A61P 27/02

(52) U.S. Cl. 514/6.5

(57) ABSTRACT

The invention is for a safe and effective method of administering an opthalmological therapeutic agent for the treatment of night blindness and improving night vision, using insulin, and chlorin e6, preparations instilled into the conjunctival sac as ophthalmic drops. Night blindness and decreased night vision is associated with retinal diseases such as dry age related macular degeneration, retinitis pigmentosa and other such related eye diseases by using insulin, chlorin e6, ketamine, and monoclonal antibodies and IGF-1. The ophthalmic preparations may be supplemented with oral intake of various retinal photoreceptors vision supporting lutein, vitamin A, Zeaxanthin, Omega 3 Oils and other nurticeuticals. They may also be supplemented with cholesterol lowering statins in the elderly with high blood cholesterol to prevent eye diseases such as AMD contributing to night vision and night blindness.

### FIELD OF THE INVENTION

Night vision described as the ability to see the objects in front of us in low light conditions and recognizable under normal light environments. The invention relates to a method for improving the night vision and treat night blindness particularly in people above the age of 50 who experience vision problems in the darkness and at night. Millions of retinitis pigmentosa, age related macular degeneration, cataract, vitamin A deficiency, diabetics; likewise, eye diseases afflicted suffer from night vision problems and night blindness. It may be the first warning sign of these impending retinal diseases where this invention can cure or curtail the underlying pathology at the same time alleviate the night blindness and improve night vision. This invention described herein also helps to enhance night vision in soldiers who have night missions without using artificial light intensifying devices. It will be of benefit to hunters, night patrol, police departments, night drivers, factory workers, and others who are engaged in work and travel after sundown.

### BACKGROUND OF THE INVENTION

The retina's light-sensing cells are rods and cones. The “cones” are specialized photoreceptor cells. They fill the central part of the retina called macula lutea. The cones sense and discriminate colors. They also see most clearly, giving us our sharp, clear, fully colored, daytime vision. The cones, however, see only ahead but not to the sides (no peripheral vision), and they do not sense extremely dim light. The cones function with and complement the functions of photoreceptors cells called the “rods.”

The “rods” like cones are specialized cells; occupy the peripheral or the off-center portions of the retina. The rods see only in black, white, and gray and do not discriminate other colors. The rods give us side or peripheral vision, in bright daylight, in very dim light, and in mixed lighting conditions such as being on a dark road at night with bright oncoming headlights shining into one's eyes. In very dim light, with no bright lights visible, a person seeing with rods, only. This is confirmed if everything appears in black, white, and gray—without colors. In a dim-light environment, with one or two bright pinpoint lights, the person sees with both the cones (for the bright light) and the rods (for the dim light). Prolonged viewing of a bright light in a dim field causes the wide pupils to close to smaller sizes and may also desensitize the rods—temporarily reducing the person's ability to see in dim light. It is here this invention described herein is useful. Thus, nighttime drivers want oncoming cars to lower their headlight beams, to prevent their being blinded. Once the bright light is gone, the pupils open up again and the rod cells resensitize, thus restoring good night vision. Most aircraft and some autos use red lights to illuminate the panel instruments because this color does not contract the pupils or overload the rods.

Categories of Night Vision Impairment and Some of the Etiological Factors of these Types

The National Institute for Rehabilitation Engineering, Hewitt, N.J. 07421 U.S.A., describes in their posting, the categories, and types of night vision impairments and blindness that are incorporated herein. There are four categories of “night vision impairment” or “night blindness.” The majority of affected individuals are non-disabled people having only limited vision problems, such as being unable to drive safely at night, work at night job properly, go hunting, or bump into things at home and so on. Luckily most of these night visions suffers can be helped, using here in described inventive method. A significant minority, however, have more serious night vision problems, “night blindness” rather than “impaired night vision.” A large number of these patients helped, even though not all can be restored to “normal” night vision with prescription glasses. The use of the herein described inventive ophthalmic drops, improve even more the night vision along with use of corrective eyeglasses. The categories of night vision impairments are:

Category 1: blurred vision in dim light with clear sharp vision in moderate and bright light: These patients see clearly in daylight but with blurred vision at night or in semi-darkness and do not complain the dim or glaring lighting conditions. Most of these patients with night vision impairments aged 40's or 50's and above, can affect one or both eyes. Prescription glasses for presbyopic due to aging prescribed especially for night driving or outdoors night vision at distance needed with our ophthalmic drops of the invention described herein.

Category 2: blinding glare and/or halos around lights: Glare and halos around light resulting in disrupting useful night vision or blinding the driver resulting in deadly auto accidents. There are numerous patients with this condition. This glare problem can affect one or both eyes. Most of these patients are developing cataract and in some due to defect or scratch in the cornea. The lenses, clear or prescription (for giving sharpest night vision) can be “gradient tinted” so that glare can be immediately filtered out by merely moving the head and eyes slightly. Do not wear sunglasses while driving in dim light. It is possible to wear a patch on the impaired eye when driving in lighting conditions where glare and/or halo problems occurs. Prescription glasses for presbyopic due to aging prescribed especially for night driving or outdoors night vision at distance needed with our ophthalmic drops of the invention described herein.

Category 3: insufficient perception in dim light: This condition can lead to accidents while driving and at work at home. This is the condition, regularly referred to as “Night Blindness” may be caused by many oculopathies and nutriceutical deficiencies such as: vitamin A deficiency, or by a controllable disease process such as glaucoma, diabetics, AMD, or, progressive disease such as retinitis pigmentosa (which damages or kills the retina's rod cells) and cataract with or without corneal pathology. The “field-expanding” eyeglasses can help people with advanced tunnel vision in retinitis pigmentosa, but will not cure the condition or stop its progression. These conditions helped by using therapeutic agents of this inventive method described herein.

Category 4: combinations of two or more of the above conditions: It can result in the person becoming functionally handicapped. Most available treatments can lead to functional improvements but not automatically complete restoration to normal functioning vision. Most of these conditions are associated with oculopathies such as age related macular degeneration (AMD), retinitis pigmentosa, diabetic retinopathy, cataract, severe vitamin A and Lutein deficiency and such that. Our invention will help all these patients to curtail the night blindness and improve their night vision.

Although both rods and comes use similar mechanisms to convert light into vision, they function differently. As described above, the Rods do not sense color; are highly sensitive; participate in dim light perception; and can quickly become saturated with light and stop responding. That is why we hardly ever see colors in dim light. Cones, on the other hand, allow us to see colors and can adapt quickly to stark changes in light intensity. Cones rely on light-sensing molecules that bind together to make up visual pigments. The pigments ruined or split when they absorb light and must be rebuilt or recycled in order for the cone cells to continue sensing light. After exposure to light, key components of pigments called chromophores leave the receptors' cells; travel to the adjacent pigment epithelium of the retina. This transported chromophore restored by retinal pigment epithelium (RPE). Then it re-transported to the photoreceptor cells in order to resume their light sensing function.

Earlier this year, the research team removed the pigment epithelium layer in salamander retinas, so that pigment molecules could not be recycled that way. Then, they exposed the retinal cells to both bright light and to darkness. The rods no longer functioned; but the cones continued to function properly even without the eye's pigment epithelium. “Exposure to bright light destroyed visual pigments in the rods, and those cells could not recycle chromophores,” says principal investigator Vladimir j. Kefalov, Ph.D., assistant professor of ophthalmology and visual sciences, Washington University in St. Louis Medical School. By contrast, pigments in cones quickly regenerated and continued to detect light even without the pigment epithelium; so it was clear, a second pathway was involved. In the new study, Dr. Kefalov did the same experiments in cells from mice, primates, and humans; all with the same result.

To learn how cones were able to recycle pigments without pigment epithelium, these investigators, focused on a particular type of cell in the retina called Müller cells. These cells support and interact with rods and cones. The researchers treated mouse retinas with a chemical that destroyed the Müller cells; then, exposed the retina to bright light, followed by darkness. When they blocked the function of Müller cells, the retinal visual pathway could not function because cones ran out of photopigment and could not adapt to dark. This suggests the Müller cells are the key to this pathway in mammals, including humans. Cones in mice, primate, and human retinas are able to function in bright light and adapt to darkness, independently of the pigment epithelium if the Müller were functioning. This discovery be of use to improve vision when the other pathway, involving pigment epithelium, has been interrupted by injury or disease, such as age-related macular degeneration.

The photoreceptors of the retina in the eye translate light (photons) into electrical signals. Though vision is a gift, the physiological processes are somewhat simple as well as complex. Retinal, the light absorbing molecule in the outer segment of the photoreceptors (which are in contact with pigment epithelium) combines with proteins called opsin to form four types of visual pigments. Depending on the form of opsin bound, retinal absorbs different wavelengths of visible light. Retinal related to vitamin A (all-trans retinol), is synthesized from it. Retinal can form different isomers. When bound to opsin, retinal has a bent shape called the 11-cis isomer; nonetheless, when the pigment struck by light, retinal straightens to the all-trans isomer form causing it to detach from opsin. This step is the only light dependant stage. Then the photochemical step initiates a series of events in the rods and cones causing electrical impulses generated and transmitted along the optic nerve.

Rhodopsin pigment of the retina is responsible for both the formation of the photoreceptor cells and the first events in the perception of light. It belongs to the G-protein coupled receptor family and is very sensitive to light, enabling vision in low-light conditions (Litmann B J, Mitchell D C (1996). “Rhodopsin structure and function”. In Lee A G. Rhodopsin and G-Protein Linked Receptors, Part A (Vol 2, 1996) (2 Vol Set). Greenwich, Conn.: JAI Press. pp. 1-32). When exposed to light, this pigment instantly photobleaches; on the other hand, it takes approximately 30 minutes to regenerate in humans photoreceptors cells. Rhodopsin is composed of the protein moiety opsin and a reversibly covalently bound cofactor, retinal. Opsin, a bundle of seven transmembrane helices connected to each other by protein loops, binds retinal (a photo reactive chromophore), which is located in a central pocket on the seventh helix at a lysine residue. Retinal positioned horizontally in relation to the membrane and each outer segment disc contains thousands of visual pigment molecules.

About half the opsin is penetrating within the lipid bi-layer of the cell membrane. Retinal is formed in the retina from Vitamin A, and dietary beta-carotene. Isomerization of 11-cis-retinal into all-trans-retinal by light induces a conformational change (bleaching) in opsin continuing with metarhodopsin II, which activates the associated G protein transducin and triggers a second messenger cascade. Rhodopsin of the rods most strongly absorbs green-blue light and therefore appears reddish-purple, which is why it is also called “visual purple”. It is accountable for monochromatic vision in the dark. Several closely related opsins exist that differ only in a few amino acids and in the wavelengths of light that they absorb most strongly. Humans eye have four different other opsins beside rhodopsin. The photopsins found in the different types of the cone cells of the retina and are the basis of color vision. They absorb yellowish-green (photopsin I), green (photopsin II), and bluish-violet (photopsin III) light to initiate electrical signal and color perception. The remaining opsin (melanopsin) is found in photosensitive ganglion cells and absorbs blue light mainly (Rhodopsin—From Wikipedia, the free encyclopedia).

Many vertebrate animals have two or more visual pigments. Scotopsin pigments are associated with vision in dim light. In vertebrates, it is found in the rod photoreceptors of the retina; the retinal₁ forms are called Rhodopsins, and the retinal₂ forms porphyropsins. Photopsin pigments operate in brighter light than scotopsins and occur in the vertebrate cone cells; they differ from the scotopsins only in the characteristics of the opsin fraction. The retinal one (1) form called iodopsins; the retinal two (2) forms cyanopsins.

The principal event in visualization is the light instigated activation of visual pigments changes in the retinal photoreceptor cells translated into nerve impulse as light. Each of the visual pigments consists of the protein opsin bound to 11-cis-retinal (a vitamin A based component). These pigments are responsible for initiating the transformation of light into electrical signals that are conducted through the optic nerve and its connections to the centers of the brain. In humans three visual pigments are used for daytime color (photopic) vision which means normal vision during daylight when the activity of the cones in the retina enables the eye to perceive color vision, sense blue (450 nm), green (530 nm) and red (560 nm) light. The visual pigment found in rods is relegated to nighttime or scotopic vision; meaning the ability to see in poor light or in the dark and responds primarily to green light. (N. Fishkin, K. Nakanishi and N. Berova, Primary events in dim light vision: a chemical and spectroscopic approach toward understanding protein/chromophore interactions in rhodopsin, Chem. Rec., 2004, 4, 120). As a result of less light scattering, dim light vision in the red region of the spectra would impart a large biological advantage, especially in conditions such as of haze, fog and underwater. (S. Johnsen, The red and the black: Bioluminescence and the color of animals in the deep sea, Integr. Comp. Biol., 2005, 45, 234-246).

However, as the absorption by rhodopsin, the opsin-retinal complex responsible for night vision in most mammals is minute above 600 nm, and the pigment not believed to sense red light. Red light vision is thus limited to red cones. However the Red cones are 100 times less sensitive than rhodopsin in detecting light. This is due to their relative rarity, higher rate of thermal isomerization, and the higher reversibility of the red pigment complex VS apoprotein and 11-cis-retinal, making them inefficient in night vision (V. Kefalov, Y. Fu, N. Marsh-Armstrong and K. W. Yau, Role of visual pigment properties in rod and cone phototransduction, Nature, 2003, 425, 526-31). It is our intension to describe a method to enhance red light nighttime vision and enhance the vision even further with or without the use of night vision optical devices.

It is recognized that inside the compound eye of the fly (Musca, Calliphora, and Drosophila), high UV sensitivity take place from the photo stable pigment, 3-hydroxyretinol that performs as a sensitizer for rhodopsin. According to this model, the photo stable UV-absorbing pigment absorbs light quanta and transfers the energy to the blue-absorbing visual pigment (K. Kirschfeld, N. Franceschini and B. Minke, Evidence for a sensitizing Pigment in fly photoreceptors, Nature, 1977, 269, 386-390). Similar energy transfer observed between retinol and rhodopsin in Simulid males flies. The energy transfer from the carotenoid salinixanthin to bacteriorhodopsin has been experimentally shown in the eubacterium Salinibacter ruber (K. Kirschfeld and K. Vogt, Does retinol serve a sensitizing function in insect photoreceptors?, Vision Res., 1986, 26, 1771-1777. S. P. Balashov, E. S. Imasheva, V. A. Boichenko, J. Ant' on, J. M. Wang and J. K. Lanyi, Xanthorhodopsin: A proton pump with a light harvesting carotenoid antenna, Science, 2005, 309, 2061-2064).

Based on the above observations and interpretation; a photo stable derivative of chlorophyll is isolated with the rhodopsin of the deep-sea dragon fish (Stomiidae) and that this visual pigment bleached with long wavelength light absorbed primarily by the photostable chlorophyll derivative. Based on these findings, it has been suggested that the fish has evolved to use the chlorophyll derivative as a sensitizer to see red light (J. K. Bowmaker, H. J. A. Dartnall and P. J. Herring, Long wave sensitive visual pigments in some deep-sea fishes: segregation of ‘paired’ rhodopsins and porphyropsins, J. Comp. Physiol. A, 1988, 163, 685-698. R. H. Douglas, J. C. Partridge, J. C., K. Dulai, D. Hunt, C. W. Mullineaux, A. Y. Tauber and P. H. Hynninen, Dragon fish see using chlorophyll, Nature, 1998, 393, 423). It is shown that in the presence of various porphyrins, the bleaching of bovine rhodopsin in response to red light is also enhanced leading to conclude that vision enhancement by an unbleachable chlorophyll derivative might therefore be a general phenomenon in vertebrate photoreception (I. Washington, C. Brooks, N. J. Turro and K. Nakanishi, Porphyrins as photosensitizers to enhance night vision, J. Am. Chem. Soc., 2004, 126, 9892-9893. Ilyas Washington, Jilin Zhou, Steffen jockusch, Nicholas J. Turro, Koji Nakanishi and Janet R. Sparrow. Chlorophyll derivatives as visual pigments for super vision in the red. Photochem. Photobiol. Sci., 2007, 6, 775-779).

Washington, et al., whose publications findings incorporated herein, showed that living rods extracted from a salamander accumulate an exogenous chlorophyll derivative that rendered them as sensitive to red light as they were to green. Recent observations of the Erenna Siphonophore, a relative of the jellyfish, suggest that the red light vision may be widespread in the deep-sea. In spite of this evidence, no red-sensitive visual pigments have yet to be isolated from deep-sea fish (S. Johnsen, The red and the black: Bioluminescence and the color of animals in the deep sea, Integr. Comp. Biol., 2005, 45, 234-246. J. S. Levine and E. F. MacNichol, Color vision in fishes, Scient. Am., 1982, 246, 140-149. R. H. Douglas, J. C. Partridge and N.J. Marshall, The eyes of deep sea fish I. Lens pigmentation, tapeta and visual pigments, Prog. Retin Eye Res., 1998, 17, 597-636. E. J. Warrant and N. A. Locket, Vision in the deep sea, Biol. Rev., 2004, 79, 671-712).

Given the above data coupled with the observations that porphyrins of virus architectures are actively transported into mammalian cells (R. W. Robey, K. Steadman, O. Polgar and S. E. Bates, ABCG2-meadiated transport of photosensitizers-potential on photodynamic therapy, Cancer Biol. Therapy, 2005, 4, 187-194). It is hypothesized that porphyrins (chlorophyll) are transported to photoreceptors to be utilized effectively enhancing mammalian red light vision (night vision). Washington, et al., investigated this hypothesis further and their studies showed that the intravenously injected chlorophyll derivative, chlorin e6 accumulates in the eyes of mice and increases the response to red or blue light (Ilyas Washington, et al., IBID). They used water-soluble chlorophyll derivative “Chlorin e6” for localization in this study. Chlorin e6, a derivative of chlorophyll, is widely used already as a food colorant, a dietary supplement in cancer therapy and for nighttime road illumination. Chlorin e6 derivative of chlorophyll is prepared from acid or base treatment and/or transmetalation of chlorophyll a. Washington, et al. showed that the chlorin e6 injected mice eyes had three times more red fluorescence (>640 nm) but not in retinas of control mice. These findings indicated that intravenously injected chlorin e6 reaches the retina and remains many hours post administration. Similar observations made in the rabbit eye experiments (R. Haimovici, T. A. Ciulla, J. W. Miller, T. Hasan, T. J. Flotte, A. G. Kenney, K. T. Schomacker and E. S. Gragoudas, Localization of rose bengal, aluminum phthalocyanine tetrasulfonate, and chlorin e₆ in the rabbit eye, Retinal. Ret. Vit. Dis., 2002, 22, 65-74). It is known that both chlorophyll and hemoglobin are structured around a porphyrin ring, the building block of both molecules with the difference being that the core of hemoglobin is iron; whereas, the core of chlorophyll is magnesium. Each has its own unique function, and yet both are very similar in that the chlorophyll is necessary for photosynthesis and hemoglobin in the red blood cell to carry oxygen to every cell in the human body. It is this building block of porphyrins, incorporated to retina in the form of Chlorin e6 or other forms of chlorophyll as they developed, to enhance the night vision and alleviated the night blindness.

There are many causes of vision deficit and loss in the elderly. The common causes of vision loss are age-related macular degeneration, retinitis pigmentosa, glaucoma, cataract, diabetic retinopathy, and host of other diseases of the cornea, retina, systemic diseases, and the use of certain therapeutic agents (David A. Quillen, Common Causes of Vision Loss in Elderly Patients. Am Fam Physician. 1999 Jul. 1; 60(1):99-108). Reduction of rods and photoreceptors pigment responsible for the night vision is accountable for development of night blindness. For the first time, the Retinitis pigmentosa afflicted experience defective darkness adaptation or nyctalopia (night blindness) followed by reduction of the peripheral visual field (contributing to the term known as tunnel vision). The present invention describes improving the visual pigment in the rods and helps to improve night vision in all these patients.

The elderly population in the United States is growing rapidly, and by the year 2030, approximately 70 million Americans will be over the age of 65 years. Loss or diminished vision with these millions of elderly is a major health care problem. Approximately one in three elderly persons has some form of vision-reducing eye disease by the age of 65. The prevalence of retinitis pigmentosa (RP), which has the first symptom of night blindness in The United States, is about one in 4000. The worldwide prevalence of RP is about one in 3000 to 1 in 5000. Any form of vision impairment due to night blindness and decline in night vision is associated with a decreased ability to perform activities of daily living and an increased risk for depression, accidents in home and while moving or driving. Aging causes, a dramatic slowing in dark adaptation attributed to delay in rhodopsin regeneration in the retinal photoreceptors hence the older person requires extra lighting. This age-related delay in dark adaptation may also contribute to night vision problems commonly experienced by the elderly.

Color vision and contrast sensitivity perceived by the cones also affected by aging. Color vision changes cause some reduction of ability to discriminate blues and blue-greens. The yellowing of the lens believed to be responsible for this effect. The aging lens and cornea causes glare by light scattering, especially for shorter wavelengths. Contrast sensitivity shows a major age-related decline (www.agingeye.net/visionbasics/theagingeye.php. Contrast sensitivity shows a significant age-related decline. Ophthalmic Physiol Opt 2000; 20:323-34). Wave front aberrations of the cornea also increase with age, leading to poor vision quality especially when the pupil dilates as in the dark (Oshika T, Klyce S D, Applegate R A, et al. Changes in corneal wavefront aberration with aging. Invest Ophthalmol V is Sci 1999; 40:1351-5).

One of the first symptoms of retinitis pigmentosa is night blindness (Nyctalopia), which is a painless and progressive; considered as characteristic of the disease. Patients with decreased night vision and night blindness struggle with tasks at night or in dark places. There is a problem walking in dim lit rooms (e.g., movie theaters), difficulties driving in low light, sundown, misty cloudy conditions where the individual needs a prolonged period of time needed to adapt from light to dark. In the early stages, the peripheral vision loss is often asymptomatic. Gradually they develop tunnel vision. Such patients may report running into furniture or doorframes. The patients struggle with sports such as tennis, softball, football, basketball where peripheral vision is required and see flashes of light (photopsia). These symptoms also observed in elderly without the retinitis pigmentosa affliction. Hence, the use of our invention insulin, ketamine, monoclonal antiboied, IGF-1, and chlorin e6 as ophthalmic drops will be of great relief to the patients to improve the night vision at the same time cure or curtail the disease in the photoreceptors and improve the vision in the elderly.

Whether by biological or technological means, at present night vision made possible by a combination of two approaches: sufficient spectral range and sufficient intensity range. Humans have poor night vision compared to many animals, in part because the human eye lacks a tapetum lucidum (Latin: “bright tapestry”). Tapetum is layer of cells in the wall of the eye of nocturnal and deep-sea animals that reflects light back onto the retina, enhancing visual sensitivity in dim light. Light reflected by this layer is responsible for the shining eyes of cats seen when they are illuminated at night found in the eyes retinal layer of many vertebrates. This layer of cells lies immediately behind or within the retina and reflects visible light back through the retinal photoreceptors, increasing the light available to the photoreceptors contributing to superior night vision of these animals. Many of these animals are nocturnal; especially carnivores that hunt their victim at night, while others are deep-sea animals. The method described herein the invention can act a temporary tapetum lucidum, and increase the night vision and curtail the night blindness.

The tapetum lucidum is a retro reflector which reflects light directly back along the light thus increasing the quantity of light passing through the retinal photoreceptors. For example, in the cat, the tapetum lucidum lowers the minimum threshold of vision 6-fold, permitting the cat to see light that is invisible to human eyes. The human eye has no tapetum lucidum, hence no eye shine in the dark. However, in humans and animals two effects can occur that may resemble eye shine: leukocoria (white shine, indicative of abnormalities including cataracts, cancers, and other diseases) and red-eye effect. Our invention will provide the means by which human or animals can enhance the night vision without tapetum lucidum.

It is the intention of this invention to provide an artificial tapetum lucidum effect to enhance the light that falls on the retina to stimulate the visual perception in rods by using porphyrins to enhance the mammalian red light vision to enhance the night vision in human and alleviate the night blindness. At the same time, improve the photoreceptors function by neuroprotective therapeutic agents.

Besides the biological method explained in this invention, there are dozens of patents on devices to enhance the night vision and many them intended be used military and surveillance media. Existing night vision systems have many applications in everyday life especially for the military and night hunters. Perhaps the most well known use for night vision systems is by the military when performing nighttime maneuvers, search, and destroy missions. The night vision devices permit vision under very low or minimal light conditions by converting incoming infrared and/or visible light from a viewed scene to an intensified visible light image. During nighttime maneuvers, military personnel are often performing other tasks, such as piloting an aircraft or driving a vehicle, that require the freedom of their hands while they visually scan the territory.

Accordingly, night vision systems developed worn upon the head of a user, such as goggles secured directly on the head or mounted to a helmet or a visor. None of these devices enhances the vision of the individual within the eyes. The driver of vehicle in the night may need help, but cannot afford night vision devices, which are expensive and bulky-cumbersome to wear. Many of these devices have countless drawbacks, and improved natural night vision described herein can add to the benefit of these devices and enhance the nighttime avocation at the same time act as prophylactic treatment for any future impending retinal diseases and other oculopathies.

The present invention will help the military personal involved in night missions, nighttime workers, hunters, drivers, and many other employees who work the night shift to perform the desired task efficiently without affecting the physical health and avoiding the injury. Improving night vision by using our invention will prevent or reduce the auto accidents, the accidents at factories workers due to poor night vision, especially those workers above the age of 50 and make nighttime missions easily accomplished with better vision. It will improve the night vision and prevent or curtail the night blindness associated with age related macular degeneration, retinitis pigmentosa and such conditions.

### SUMMARY OF THE INVENTION

The invention is for a safe and effective method of administering an opthalmological therapeutic agent for the treatment of night blindness and improving night vision, using insulin, and chlorin e6, preparations instilled into the conjunctival sac as ophthalmic drops. Night blindness and decreased night vision is associated with retinal diseases such as dry age related macular degeneration, retinitis pigmentosa and other such related eye diseases by using insulin, chlorin e6, ketamine, and monoclonal antibodies and IGF-1. The ophthalmic preparations may be supplemented with oral intake of various retinal photoreceptors vision supporting lutein, vitamin A, Zeaxanthin, Omega 3 Oils and other nurticeuticals. They may also be supplemented with cholesterol lowering statins in the elderly with high blood cholesterol to prevent eye diseases such as AMD contributing to night vision and night blindness.

A goal of the present invention is to provide a new, useful, simple, and effective method for improving, and enhancing night vision.

It is a goal of the present invention is to provide a new, useful, simple, and effective method for improving, and enhancing night vision by improving the photoreceptors function.

Another goal of the present invention is to present a safe and effective treatment, for night vision deficiency and night blindness associated with various oculopathies including retinal photoreceptors afflictions.

Another Goal of the present invention is to present a safe and effective treatment for retinitis pigmentosa, age related macular degeneration, diabetic retinopathy, likewise eye disease afflicted patients to curtail or cure the underlying pathology that contributes to the night blindness and improve night vision.

Goal of the present invention is to make available a safe and effective treatment with or without the use of any nighttime goggles for enhancing night vision.

A further goal of the present invention is to provide a safe and effective treatment inexpensively to prevent night blindness, which is prevalent after the age of 50.

An important function of this invention is to allow unobstructed movement of the eyes while at the same time provide therapy for night blindness and improve night vision in nighttime workers and soldiers who are engaged in night missions.

Yet another purpose of the present invention is to provide an inexpensive method to improve night vision and to reduce the incidence of night blindness in night vehicle drivers and prevent vehicle driven accidents.

Yet another goal of the present invention is to improve night vision without the use of light intensifying optical system devices, which are bulky and cumbersome to wear besides being expensive.

A further aim of the present invention is to provide a safe and effective therapy and remedy night blindness and improve night vision, inexpensively that can be self-administered and be used when needed.

It is another purpose of this invention is to offer ophthalmic drops containing insulin, chlorin e6, IGF-1 along with NMDA blocker ketamine, and such related drugs, to prevent the glutamate excitotoxicity damage to the photoreceptors.

It is another purpose of this invention also to neutralize autoantibodies involved in retinitis pigmentosa causing night blindness due to autoimmune related diseases afflicting the eyes by using monoclonal antibody Etanercept, to prevent the night blindness and improve the night vision.

### BRIEF DESCRIPTION OF THE DRAWINGS

The embodiments of the eye components in the drawings are not necessarily to scale, but stress instead being placed upon visibly and clearly so as to illustrate the principles of the present invention that shows how conjunctional sac instillation of therapeutic agents described herein are delivered to the retina, to enhance the night vision, in which;

FIG. 1 is a diagram of the longitudinal section of the eye showing conjunctival sac containing combination ophthalmic drops of this invention.

FIG. 2 is a drawing of the longitudinal section of the eye showing the structures involved in the production and drainage of aqueous humor, which transports insulin, and chlorin e6, ketamine and monoclonal antibodies and other combination adjuvant therapeutic agents.

FIG. 3 is a diagrammatic presentation section of the anterior part of the eye presentations with rich vascular plexus, which transports the therapeutic agents to the retinal photoreceptors.

FIG. 4 is the diagrammatic presentation showing the vascular arrangement of the choroid surrounding the retina, which supplies nutrition including oxygen to the outer segments of photoreceptors and delivers therapeutic agents of our invention.

FIG. 5 is the diagrammatic presentation 500 showing the histology of the choroid with rich vascular plexus, retina, and their blood supply.

FIG. 6 is the diagrammatic presentation 600 showing the histology of the retina, its relation to the blood supply, and the route of transfer of therapeutic agents to photoreceptors.

FIG. 7 is the diagrammatic presentation 700 showing the conjunctival fornix and the route of drainage of therapeutic agents to the nose.

### DETAILED DESCRIPTION OF THE INVENTION

The ophthalmic drops or preparations used to treat decreased night vision and night blindness and other associated eye diseases should be stable, dissolved, or solubilized ensuring the preparation is safe and effective with opthalmological standards in place. The term ‘stable,’ means physical, rather than chemical stability with no crystallization and/or precipitation in the compositions, when the preparation is stored at a refrigerated or room temperature. The preparation encounters lacrimal secretions when the preparations instilled to the conjunctival sac and the cornea and should not react with it. The label ‘dissolved,’ ‘dissolving,’ ‘solubilized’ or ‘solubilizing’ means that an ingredient is substantially solubilized in the aqueous composition and in the lymphatic and vascular circulation without the particulate, crystalline, or droplet form in the composition when absorbed and transported. The phrase “ophthalmic” “opthalmological” acceptable,’ refers to those therapeutic, pharmaceutical, biochemical and biological agents or compounds, materials, compositions, and/or dosage forms suitable for use in a mammalian eye without undue toxicity, irritation, allergic response, or other problem or complication, commensurate with a reasonable benefit/risk ratio. The expression ‘safe and effective,’ as used herein, means a concentration and composition, that the concentration and composition is sufficient to treat without serious local or systemic side effects.

The term “ocupopathies” means all diseases affecting the retina mail, and also includes afflictions of the eyelids, eye ball, lens, optic nerve, uveal system with choroid, cranial nerve and the ocular muscles they supply, neuro-muscular structures of the eyes, membranes covering the eyes and their function. In general, it refers to retinal and uveal diseases, unless specified. The term “drop” “drops” means the therapeutic agents delivered or instilled to conjunctional sac by a dropper or plastic squeeze bottle drop by drop. “Night blindness” and “night vision” “vision problems” are interchangingly used. The terms “treat,” “treating” and “treatment” “curtail” envisage reducing the seriousness of the disease, or retards or slows the progression of the disease.

Before the explanation and description of the disclosed diagram of the present invention in detail, need to understand that the invention is not limited in its application to the details of the particular examples and arrangements shown in the diagram. As enumerated above and narrated below; this application has been filed in order to disclose: insulin, and cholin e6, with or without ketamine, monoclonal antibodies insulin like growth factor (IGF-1) that have high therapeutic activity in modulating the physiological functions including retina with photoreceptors involved in night vision and night blindness. They effectively curtail or cure the pathological states, which leads to various diseases of the Photoreceptors involved in perception of light.

The following diagrams describe the structure of the eye, and explain the route of movement, transportation, and diffusion of insulin, chlorin e6, and various known adjuvant therapeutic agents instilled in the conjunctival sac and administered orally, parenterally by injection, infusion, or implantation. In the following detailed, description of the invention, reference is made to the drawings, which reference numerals refer to the like elements. It is understood where other embodiments may be utilized that the structural changes may be made without departing from the scope and the spirit of the invention described herein.

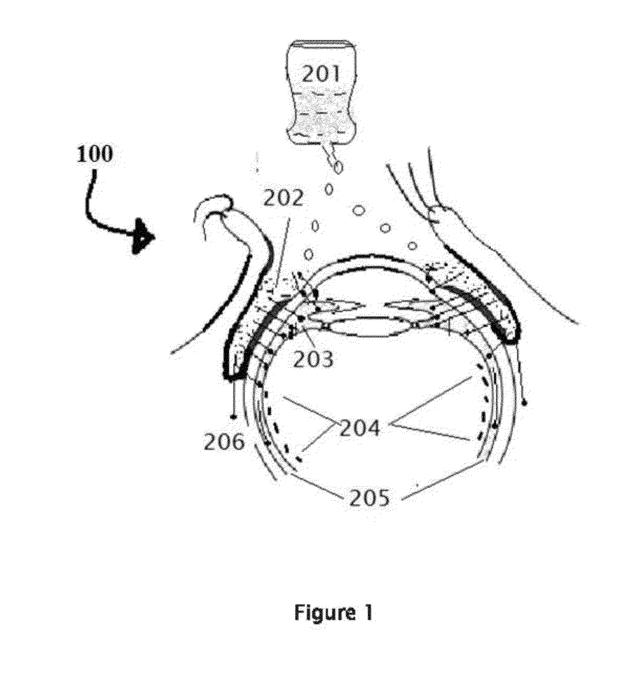

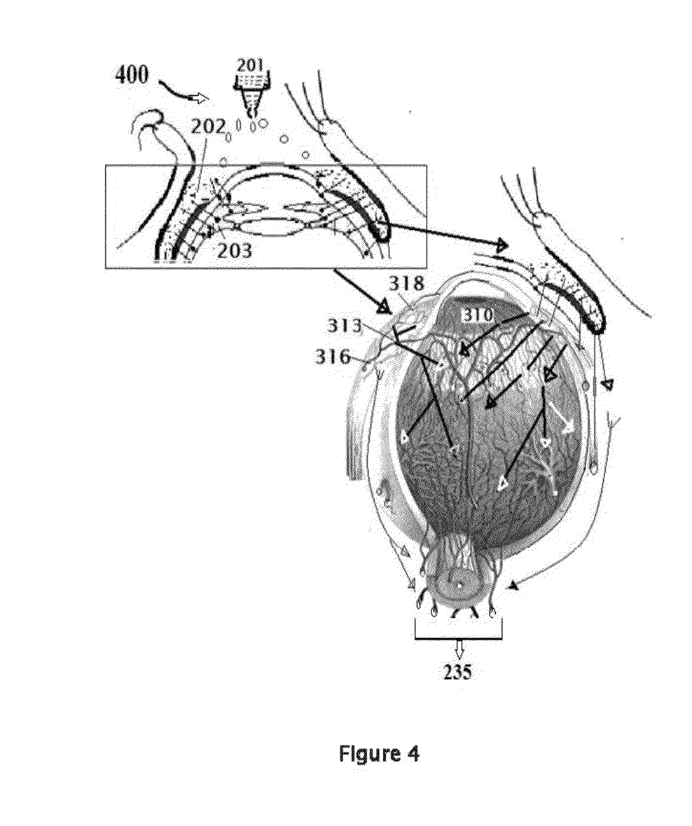

FIG. 1 is the diagram of the longitudinal section of the eye 200 showing conjunctival sac 202 containing the instilled insulin, and chlorin e6 drops with or without adjuvant therapeutic agents described in this invention. The other therapeutic agents introduced through a dropper 201 and their passage to iridocorneal angle, anterior and posterior chambers, iris, ciliary body, and processes 203, choroid, and the anterior segment of the retina 204 which the other therapeutic agents contains photoreceptors rods. The photoreceptors are affected by the night blindness and other associated retinal diseases (drumstick markers). Note that the ophthalmic insulin and chlorin e6 eye drops and other therapeutic agents pass on to the choroid 205 adjacent to the retinal pigment epithelium and retinal photoreceptors. This delivers the therapeutic agent chlorin e6 to the afflicted rods, which participates in improving the night blindness. The therapeutic agent's passes through the episcleral plexus of blood vessels to the periphery of the sclera 206 where the agents reabsorbed (see FIG. 4 #235) the agents circulate back into the choroid and retinal blood vessels (BV).

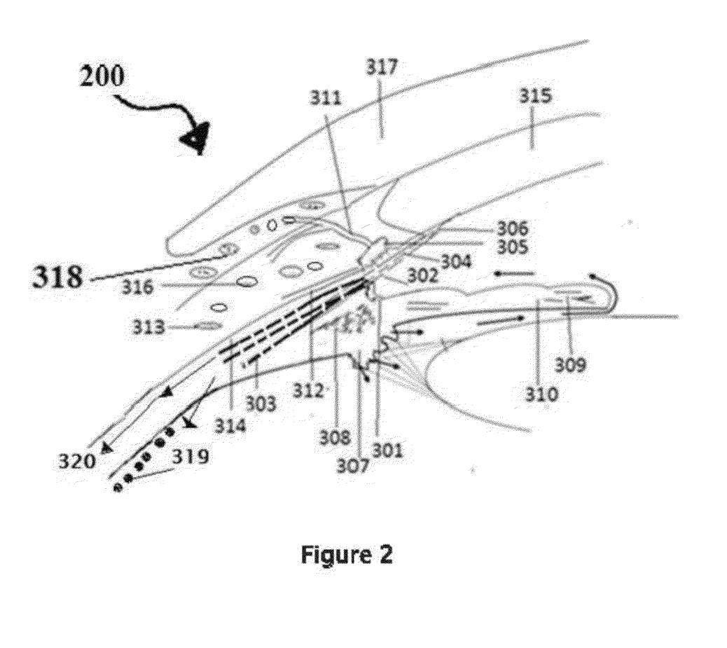

FIG. 2 is the drawing of the longitudinal section of the eye 200 showing the structures involved in the production and the drainage of aqueous humor. The aqueous humor and associated structures pick up the therapeutic agents from the conjunctional sac insulin and chlorin e6, used in the treatment of decreased night vision and night blindness and other associated and/or etiological retinal oculopathies, described in this invention. The insulin, and chlorin e6 circulates through various sites of action where the therapeutic agents reach their ultimate site of action—to the retinal rods and cones (arrows) with ease involved in night vision and blindness. These therapeutic agents enter the anterior chamber aqueous humor through the episcleral arteriovenous plexus 313,316. Then pass through the uveoscleral meshwork 301, corneoscleral meshwork 302, juxtacanalicular or cribriform trabecular meshwork 304, Schlemm's canal 305, corneal endothelium joining the trabecular meshwork 306, longitudinal 303, and circular fibers of the ciliary muscles 308; muscle fibers of the iris 309, 310, scleral sinus vein 311, scleral spur 312, scleral veins 313,316, suprachoroidal space between choroid and sclera 314. The cornea 315, sclera 316, and cornea-scleral junction participate in therapeutic agent's circulation or transportation takes place with the help of the blood vessels and aqueous humor. The conjunctival sac 317 (fornix) where the insulin, and chlorin e6, and other therapeutic, pharmaceutical, biochemical, and biological agents, or compounds are deposited to be transported (arrows) to the retina through the uveal system such as ciliary body 307, choroid, choroid plexus projecting from the ciliary body 307, trabecular mesh work, and irido-scleral angle 301. The choroid and suprachoroidal space 314, 320 with extensions of pia arachnoid lamella from the optic nerve (Shantha T R and Bourne G H: Histological and histochemical studies of the choroid of the eye and its relations to the pia-arachnoid mater of the central nervous system and Perineural epithelium of the peripheral nervous system. Acta Anat 61:379-398 (1965)) plays an important role in transporting the insulin and chlorin e6, and various other therapeutic agents (arrows) to enhance the night vision and treat the night blindness and other associated oculopathies afflicting the retina 319.

This diagram illustrates the ease in which the insulin and chlorin e6, and other selected therapeutic agents reach the afflicted night blindness contributing rods and other associated oculopathies from the site from the conjunctival sac 317 (arrows). From conjunctival sac 317, the therapeutic agents enter into the anterior chamber, cornea, and corneal endothelium 304, 306 (Shantha T R and Bourne G H. Some observations on the corneal endothelium. Acta Ophthalmologica 41: 683-688: 1963), trabecular meshwork 301, 302, and ciliary body 308 passing through the sub and inter conjunctival blood vessel plexus of the eye 313, 316, 318, choroid 320, suprachoroidal space 314 where they reach their destination 319 to have therapeutic effect on the retinal rods involved in night vision, night blindness and other associated oculopathies. The arrows indicate the site of entry and the circulation of the insulin and chlorin e6 with other therapeutic agents from the conjunctival sac where they exert their therapeutic effect in the treatment of night blindness and other associated oculopathies. It is likely that the concentration of chlorin e6 increases in the outer segment of the rods and cones, its uptake and effect further augmented by insulin.

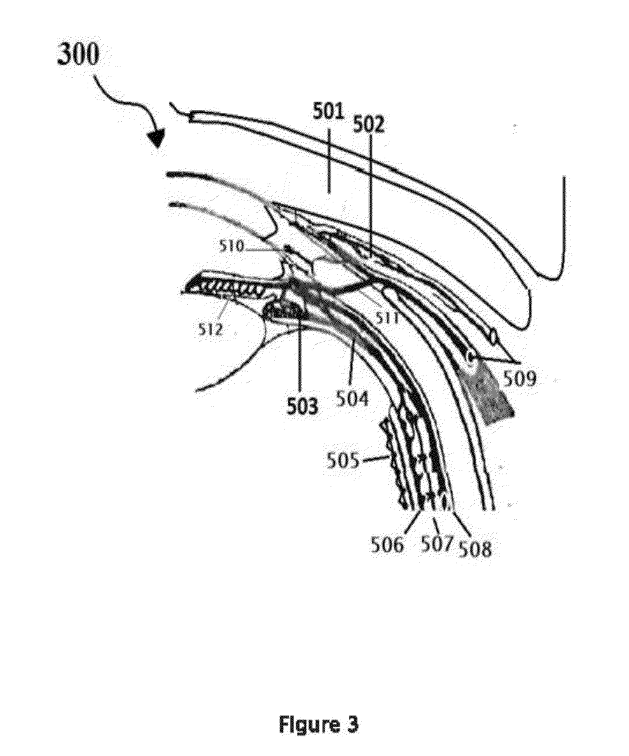

FIG. 3 is a diagrammatic section of the anterior part of the eye 300 presenting the rich vascular plexus, in which the BV of the vascular plexus are responsible for transporting the insulin, cholin e6, and other therapeutic agents from the conjunctival sac 501 to the site of night vision and blindness and other associated oculopathies 505. The rich vascular plexus 502 under the conjunctiva of the eyeball participates and helps to transport the therapeutic agents from the conjunctival sac 501 to retina of this invention. From here the therapeutic agents transported to intrascleral 511 blood vessels and canal of Schlemm 510 with the arterio-venous connection, various vascular plexus of the iris 512, iridocorneal angle, ciliary body with the ciliary processes 503, finally passes to the choroid vascular plexus 504, 507. From choroidal BV, supra and inter choroidal space 508 (see FIG. 2—314, 320), the therapeutic agents pass through the choriocapillaris and Bruch's membrane, retinal pigment epithelium 506, then the therapeutic agents reach the outer segments or the base of the rods and cones of the retina 505, the site of the night vision, night blindness and other associated oculopathies. Note the rich vascular plexus of the iris 512, choroid, ciliary body 503, which communicates with the subconjunctival BV 502, suprachoroidal space 508, and choroidal vascular network 504,507. This choroidal vascular net delivers insulin and cholin e6 and other anti night blindness and other associated oculopathies therapeutic agents to various structures between the ciliary body and the iridoslceral angle and scleral—corneal space, and supra scleral network of vascular plexus 509 finally reaching the retina. This diagram shows the vascular network under the conjunctival sac and under the insertion of the superior oblique and other external ocular muscles 509 that delivers the insulin, IGF-1 and cholin e6 to choroid and the photoreceptors. The therapeutic agents delivered to the site of night vision and blindness, i.e. the outer segment of the photoreceptors and other associated oculopathies through various vascular plexus to the afflicted rods and enhance night vision and alleviate the night blindness, thus improve the vision at nighttime.

FIG. 4 is the diagrammatic presentation 400 showing the vascular arrangement of the uveal track, the middle layer of the eyeball. It is divided from front to back into iris 310, ciliary body 203, and the choroid (arrows). The choroid covers the entire retina and is involved in the transfer of insulin and cholin e6 and other therapeutic agents of this invention to the retina the sites of the night vision, to enhance the night vision, and alleviated or reduce the night blindness. These three structures of the uveal system are vascular, that these structures communicate with the subconjunctival 318 and scleral vessels 313,316 and the muscle blood vessels (see FIG. 3—509). The entire uvea is drenched with circulating aqueous humor and the CSF from the optic nerve through the lamina cribrosa, entering supra-choroidal and inter-choroidal space (Shantha T R and Bourne G H: Arachnoid villi in the optic nerve of man and monkey. Experimental Eye Research, 3:31-35 (1964)). The insulin, chlorin e6 and the adjuvant therapeutic agents 201 from the conjunctival sac 202 are transported to the sub conjunctival arterio-venous plexus 318 inter and epi scleral veins 313,316 to reach the uveal system.

Then the therapeutic agents transported to the uveal vascular plexus (multiple drumstick and plain arrows). This rich vascular plexus transports the therapeutic agents to the retina where the agents are immediately adjacent to the choroid situated on the Bruch's membrane, retinal pigment epithelium, and outer segment of the rods as well as cones in the macula lutea. The blood vessels of the uvea are involved in the health of the retina by transporting and by providing proper nurticeuticals; at the same time the metabolites removed from the photoreceptors. In the same fashion, they carry insulin, cholin e6, and the other adjuvant therapeutic agents they deliver to the retinal rods and cones; the site of night vision and night blindness, and other associated oculopathies. This diagram shows how efficiently the insulin and cholin e₆ and the other therapeutic agents from the conjunctival sac 202 are absorbed, and transported to the subconjunctival, scleral, and uveal vascular plexus 318, 313,316. Then delivered to the uveal system (arrows) including iris 310, choroid 313 (arrows) and then to the retina, the site of night vision and blindness and other associated oculopathies. Arrows points to the spread of therapeutic agents from the conjunctival sac to the rich uveal vascular network. Arrows shows that some of the therapeutic agents are transported to the supra scleral space where the agents may be transported back through the penetrating short and long arterio-venous network 235 on the optic nerve (arrows) and posterior surface of the sclera (Based on Grays Anatomy diagram 7.255 on the histology of the eye).

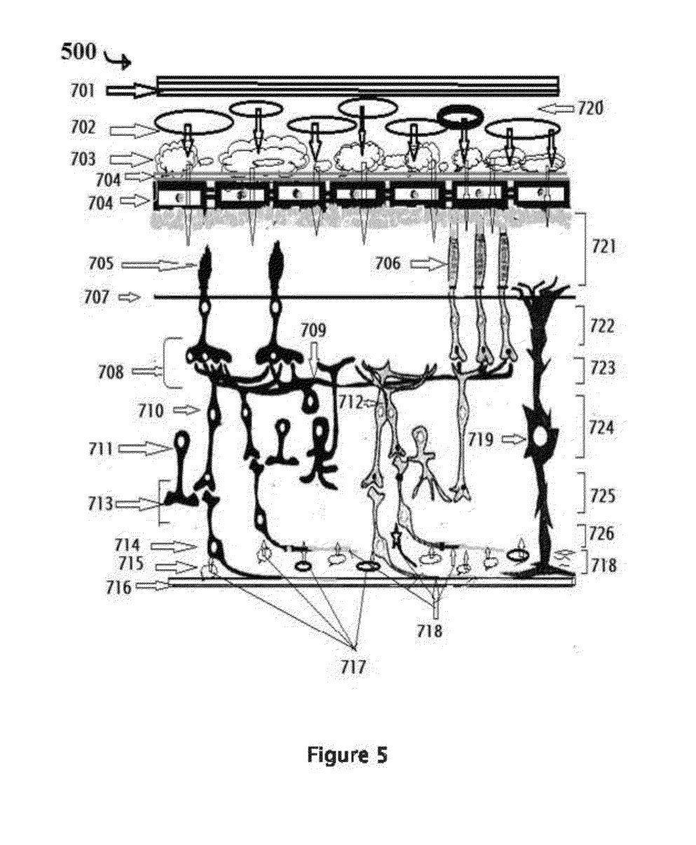

FIG. 5 is the diagrammatic presentation 500 showing the histology of the retina in relation to the blood supply and delivery of therapeutic agents of this invention to the retina. Therapeutic agents of this invention use insulin, cholin e6 and other adjuvant therapeutic agents that reach the rod and cone photoreceptors cells involved in the decreased night vision and night blindness and other associated oculopathies. It shows sclera 701, large choroidal blood vessels 702, fenestrated choriocapilareis 703 which the choroidal blood vessels delivers the insulin, cholin e6, and the other therapeutic agents (indicated by multiple large and the small arrows directed downwards towards rods and cones) which the choroidal blood vessels carry oxygen and nutriceticals, through the noncelluar Bruch's membrane 704. The Bruch's membrane acts as a interface between the pigment epithelium 704 and fenestrated choriocappillaries 703. This separates retinal pigment epithelium form the choriocapilaries 703 and prevents their invasion into retina. In way it is outer retinal barrier preventing the choriocapillaries reaching the retina. The cones 705 are not in intimate contact with the retinal pigment epithelium 704. The rods are in close contact with the retinal pigment epithelium where they brush the border 704, hence receive the therapeutic agents easily. The outer limiting membrane 707 formed by the Müller cells 719 separates the photoreceptors outer segments from the rest of the retina which the separation may prevent the transfer of components from extracellular space of the photoreceptors to the rest of the retina.

In the same fashion, the therapeutic agents get concentrated as they are transported from choriocapillaries towards the outer segment of the photoreceptors which is the site of decreased night vision and night blindness and other associated ocular pathology. Note the outer plexiform layer 708 and horizontal cells 709 are the laterally interconnecting neurons in the outer plexiform layer of the retina. These neurons modify and integrate the signals from the rods and cones where the rods and the cones are responsible for allowing eyes to adjust allowing the patient can see equally in bright and dim light conditions. They help to integrate and regulate the input from multiple photoreceptor cells. The bipolar cells 710, 712 situated between photoreceptors (rods 706 and cones 705) and ganglion cells 714. The therapeutic agents from the conjunctiva do not reach these cells in high concentration due to the presence of outer limiting membrane and paucity of vascular network. They get very low doses of the therapeutic agents through the central artery of the retina from the systemic circulation. They act, directly or indirectly, to transmit signals from the photoreceptors to the ganglion cells. Amacrine cells 711 are the interneurons (40 types are recognized) which the amacrine cells are responsible for 70% of the input to retinal ganglion cell 714. The bipolar cells 710, 712 are responsible for the other 30% of input to the retinal ganglia.

They, along with the ganglion cells, do receive the therapeutic agents of our invention through the central artery of the retina through systemic circulation, though in small quantities. The inner plexiform layer 713, ganglion cell layer 714 (where the ganglion cell layer receives the signals from the rods and cones). The inner retinal blood vessels 717 supply oxygen and nutrients to the inner part of retina in which the inner retinal blood vessels are shown by multiple short arrows pointed towards outer side of the retina. The optic nerve fibers 718 derived from the ganglion cells 714 relay the photoreceptors signals to the CNS.

Note the Müller cell 719 extends inward and contributes to the inner limiting membrane 716 separating the vitreous from the retina and the outer limiting membrane 707. This isolates the sensitive outer segment of the photoreceptors cells of the retina from the rest of the retina. Experimental studies have shown that the Müller cells play an important role in regeneration of photopigment in cones besides supporting and interacting with rods and cones. Experiments on rats show that without Müller cells, cones ran out of photopigment and could not adapt to dark indicating the role these cells play in photopigment production. The arrows from choroid indicate the rich vascular supply to the outer segments of the photoreceptors (compared to the rest of the retina), which outer segments receive the therapeutic agents from the conjunctiva compared to the paucity of BV from the retinal inner BV 717. This diagram shows the insulin, cholin e6 and other therapeutic, pharmaceutical, biochemical and biological agents or compounds from conjunctiva and uveal—chorid blood vessels have easy access to rods 706 and cones 705 outer segments in improving the night vision and the treatment of night blindness as well as other associated oculopathies.

The insulin, cholin e6 and other therapeutic agents of our invention are transported by the aqueous humor through the suprachoroidal space where the agents permeate to the space between the retinal pigment epithelium and the photoreceptors. The inner limiting membrane 716 is the boundary between the retina and the vitreous body. The inner limiting membranes formed by astrocytes and the end feet of Müller cells 719. This membrane separates the vitreous humor by a basal lamina and can be akin to Bruch's membrane below the choriocapillaries. There may be some seeping of aqueous humor from ciliary epithelium and zonule fibers containing insulin, cholin e6 and other therapeutic agents ooze between these two structures through this basal lamina. This transport or soaking with therapeutic agents through this route has to be minimal at best. If it does seep, the concentration is mostly at mid and anterior part of the lower segment (between 5-7 o'clock positions) of the retina due to gravitational pull where the pathology of night blindness and other associated oculopathies are prominent. The mid and anterior parts of the retina are the main parts affected by the night blindness.

This diagram 500 also shows various histological layers of the retina. They are: the layer of retinal pigment epithelium 704, layer of rods and cones 721, outer nuclear layer 722 made up of nuclei from rods and cones, outer limiting membrane 707 formed by Müller cells, outer plexiform layer 723 made up of synapses between the rods and cones with horizontal and bipolar cells. The inner nuclear layer 724 made up of bipolar and amacrine cell nuclei, inner plexiform layer 725 formed by synapses between the ganglion cells 714, 726, and the process of cells from the inner nuclear layer. The nerve fiber layer 718 formed by the axons of the ganglion cells grouped to become the optic nerve where the nerve fiber leaves the eye at the optic disc to lateral geniculate bodies then to the occipital cortex. The inner limiting membrane 716 made up of Müller cells expanded inner feet and astroglia. The diagram shows how each retinal layer is in touch with the blood vessels with their supply of nurticeuticals, oxygen, insulin, cholin e6, and other therapeutic agents used in the treatment of night blindness and other associated oculopathies. It is clear that the outer segment of the photoreceptors get the most exposure to the therapeutic agents compared to other functional units of the retina by the use of ophthalmic drops as described in this invention. The therapeutic agents delivered to treat night vision and night blindness with insulin, and chlorin e6 reach the outer segments to improve the night vision with regeneration and restoration of night vision pigments.

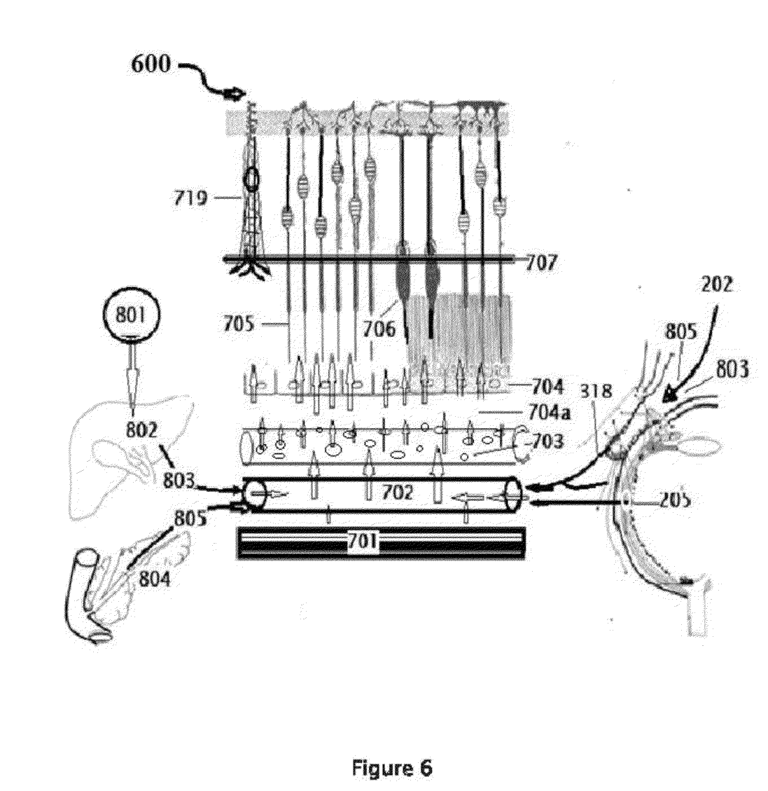

FIG. 6 is the diagrammatic presentation 600 showing the histology of the external layers of the retina including photoreceptors as described in FIG. 5 which receive the therapeutic agents of the inventions described herein. This illustrates the relation to the blood supply and the delivery of therapeutic agents of our invention to the outer segment of the rods. Our invention of insulin, cholin e6 and other therapeutic agents are transported through the systemic blood supply also from the conjunctival sac of the eyes to reach the rods and cones photoreceptors cells involved in night vision and the pathogenesis of the disease of night blindness and other associated oculopathies. It shows sclera 701, large choroidal blood vessels 702, fenestrated choriocapillareis 703 through which the capillaries deliver the therapeutic agents insulin and cholin e6 805 and/or insulin and cholin e6 like photopigment enhancer growth factors 803 from the ophthalmic drops 202. Liver 802 makes IGFs from growth hormone 801 from the pituitary gland. The pancreas 804 produces insulin, transported all over the body including to the retina 805. The insulin, cholin e6 chlorophylls 805 and other chlorophylls derivatives enters the circulation through the portal circulation, reaches the retina through the systemic circulation 805 when the therapeutic agents taken orally. The oral chlorin e6 and other ocular nutraceuticals such as lutein, carotene enters the eyes absorbed from the intestines through systemic 805 and portal 803 circulations. Hence, it is important to put on the patients with night blindness on the regimen of lutein and carotene rich diet to boost the ophthalmic drops of this invention. The ophthalmic drops 202 of insulin and cholin e6 805 and IGFs 803 are absorbed by the subconjunctival blood vessels 318 and choroid 205 as described in above figure explanations.

The growth hormone from the pituitary gland converted to IGFs (mostly in the liver) where the IGFs circulated to reach all over the body including choroidal BV 702, choriocapillares 703, central artery of the retina and ultimately, reaches the retina. The pancreas produces the insulin 805 and cholin e6 absorbed by the intestinal circulation 804 reaches the choroidal BV 205 where it also, reaches the retinal photoreceptors 705, 706. The insulin, cholin e6, and IGF-1 from the choroidal BV 702 pass to the fenestrated choriocapillares 703 which the choriocapillaries are leaky. The leaked fluid from the inside to extracellular space 707a accumulates at acellular Bruch's membrane 704a. From this space, the insulin, cholin e6, and IGF-1 passed through the retinal pigment epithelium (RPE) 704 to reach the outer segments of the photoreceptors 705, 706.

The extracellular fluid is bound by retinal pigment epithelium (RPE) and the external limiting membrane 707 and is formed by the Müller cells 717. Thus, our method transports the chlorophyll and its derivative chlorin e6 that delivered with the help of insulin to the outer segment of the rods that are in direct contact with the pigment epithelium. This porphyrin based chlorophyll and its water-soluble depravities delivered to the rods to improve the night vision and cure or curtail night blindness. The big and small arrows show the directions of the flow of insulin, e6 chlorin; other similar chlorophyll based therapeutic agents and IGF-1 from the conjunctival sac 202 where there is the systemic circulation from liver and pancreas. The arrows from the choroid indicate the rich vascular supply to the outer segments of the photoreceptors which the photoreceptors receive the therapeutic agents from the conjunctiva compared to the paucity of BV from the retinal inner BV. This diagram shows that the therapeutic, pharmaceutical, biochemical, and biological agents or compounds from conjunctiva and chorid blood vessels have easy access to rods 706 and cones 705 in the treatment of night vision and night blindness and other associated oculopathies. The therapeutic agents are also transported by the aqueous humor and capillary fluid through the suprachoroidal space where the agents permeate to the space between the retinal pigment epithelium and the photoreceptors. IGF-1 not used in the ophthalmic solutions for patients with wet AMD, diabetic retinopathy, ocular tumors, oculopathies associated with angiogenesis etc.

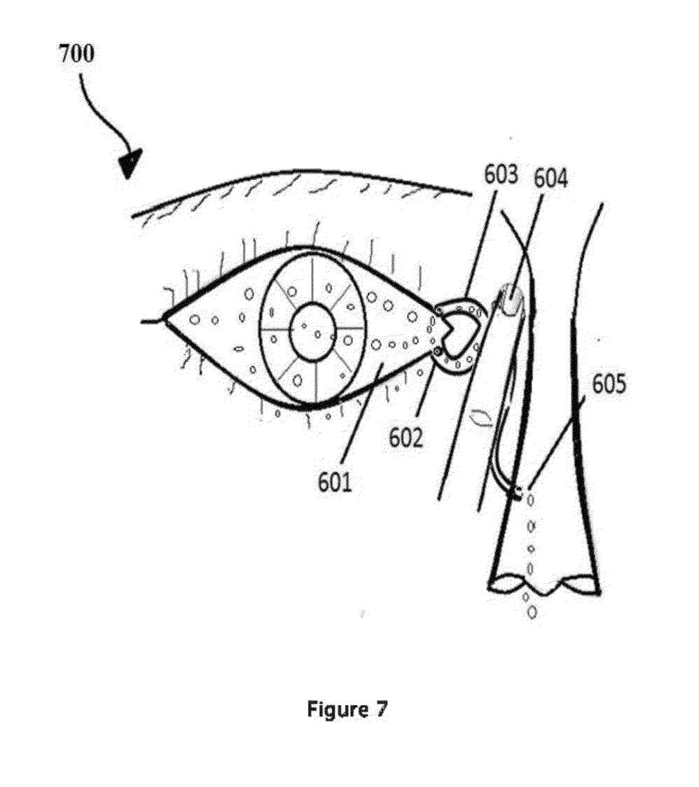

FIG. 7 is the diagrammatic presentation 700 showing the route of drainage of the lacrimal fluid and therapeutic agents shown as bubbles from the conjunctival fornix (sac) 601 to the nasal mucosa 605 and illustrates a method to prevent the therapeutic agents of this invention from entering the nasal mucosa. A simple method applying the finger pressure 604 at the medial eye angle and nasal junction. The location of the lacrimal punctum, canaliculi 602, 603 and lacrimal sac with a finger 604 will prevent the therapeutic agents drainage to the nasal cavity and the nasal mucosal absorption 605, and their associated systemic adverse effects.

Route of Insulin, Chlorin e6, and Other Therapeutic Agents Such as IGF-1, Ketamine, Monoclonal Antibodies (Etanercept™) Etc. Transported Through the Choroid to Reach Photoreceptors from the Conjunctival Sac for the Treatment of Night Blindness, to Improve Night Vision, at the Same Time Treat the Underlying Etiologies Such as Age Related Macular Degeneration (AMD), and Retinitis Pigmentosa.

To understand how the therapeutic agents from the conjunctival sac of the eye reach the retinal pigment epitheliums (RPE) and retinal photoreceptors as described in FIGS. 1 to 6; knowledge of the histology and the blood supply of this region, which are responsible for transporting these drugs to the site of pathology and to restore and enhance the natural physiological function.

The choroid, also known as the choroidea or choroid coat, is the vascular layer of the eye, situated between the retina and the sclera. The human choroid is thickest at the rear of the eye (at 0.2 mm), while in the outlying areas it narrows to 0.1 mm. The choroid provides oxygen and nourishment to the outer layers of the retina. Along with the ciliary body and iris, the choroid forms the uveal tract.

The structure of the choroid is generally divided into four layers:

Haller's layer—outermost layer of the choroid; consisting of larger diameter blood vessels; multiple thin layers of squamous cells, an extension of pia-arachnoid mater from the optic nerve, and supra-inter-choroidal space between and above these cell layers. (Shantha T R and Bourne G H: Histological and histochemical studies of the choroid of the eye and its relations to the pia-arachnoid mater of the central nervous system and Perineural epithelium of the peripheral nervous system. Acta Anat 61:379-398 (1965). Shantha T R and Bourne G H: Arachnoid villi in the optic nerve of man and monkey. Expt Eye Res 3:31-35 (1964)).
Sattler's layer—layer of medium diameter blood vessels;
Choriocapillaris—layer of capillaries; made up of fenestrated BV. They participate in delivering therapeutic agents of our invention besides supplying the nutrients and oxygen to the outer segments of the photoreceptors;
Bruch's membrane (synonyms: Lamina basalis, Complexus basalis, Lamina vitra)—innermost acellular layer of the choroid, situated between the choriocapillaries and retinal pigment epithelium.

There are two sources of blood supply to the mammalian retina: the central retinal artery and the choroidal blood vessels (FIGS. 3, 4). The choroid receives the maximum blood flow (65-85%) compared to any component of the eye; it is vital for the maintenance of the outer retina (particularly the photoreceptors) and the remaining 20-30% flows to the retina through the central retinal artery from the optic nerve head to nourish the inner retinal layers. The central retinal artery has 4 main branches. There are two circulations of the eye: the retinal and uveal, supplied in humans by posterior ciliary arteries, originating from the ophthalmic artery. The arteries of the uveal circulation, supplying the uvea and outer and middle layers of the retina, are branches of the ophthalmic artery and enter the eyeball without passing with the optic nerve. The retinal circulation, on the other hand, derives its circulation from the central retinal artery, also a branch of the ophthalmic artery, but passing in conjunction with the optic nerve. They branch in a segmental distribution to the end arterioles and not anastomose. This is clinically significant for diseases affecting choroidal blood supply. The macula responsible for central vision and the anterior part of the optic nerve are dependent on choroidal blood supply.

The blood supply to the eye derived from the ophthalmic branch of the internal carotid artery. One of the branches of this artery will become the central retinal artery of the retina entering the retina through the optic nerve, and the rest splits to multiple small sized arteries on each side of the optic nerve (FIG. 4). These vessels divide into 2 long posterior ciliary arteries and 12-20 short posterior ciliary arteries that enter the eye immediately adjacent and around the optic nerve (FIG. 4—#235). The short posterior ciliary arteries directly supply the choroid. The long posterior ciliary arteries travel in the suprachoroidal space anteriorly then supply the choroid anteriorly via recurrent branches. Blood in the choroid circulates through the choriocapillaries and larger vessels of the choroid drain into 4-6 vortex veins (FIG. 4). The vortex veins emerge just posterior to the equator in eye quadrants (see FIGS. 3, 4). The superotemporal and superonasal vortex veins will drain into the superior ophthalmic vein. The inferonasal and inferotemporal vortex veins will drain into the inferior ophthalmic vein and will eventually exit via the cavernous sinus. Hence, some of the therapeutic agents absorbed from the conjunctional sac reach the hypothalamus, basal part of the frontal lobe, prefrontal cortex, entorhinal cortex, and brain stem. The vortex veins also anastomose with the anterior ciliary veins, which normally carry blood from the anterior ciliary muscle.

The circulation of the anterior portion of the eye shows an intricate vascular anastomotic system that essentially joins the anterior ciliary circulation and the long posterior ciliary circulation in three interconnected arterial-venous circles (FIGS. 3, 4);

1) An episcleral circle where episcleral vessels join,
2) An intramuscular ciliary circle, and
3) The major arterial iris circle (circumferential vessels in the ciliary body) with choroidal BV.

The anterior ciliary vascular system joins subconjunctival and episcleral vessels at the limbus of the eye (FIGS. 2, 3, 4). These blood vessels are involved in the delivery of therapeutic agents Insulin, and chlorin e6 of our invention to photoreceptors to treat night blindness and to improve night vision and other oculopathies from the conjunctival sac.

The Insulin, Chlorin e6 and Other Therapeutic Agents Such as IGF-1, Monoclonal Antibodies, Ketamine Therapeutic Agents Administered into the Conjunctival Sac Transported Through Two Types of Blood-Retinal Barriers Namely: 1) Inner Blood-Retinal Barrier (FIG. 5) and, 2) Outer Blood-Retinal Barrier (FIGS. 5, 6).

1. The inner retinal barrier is formed by the central artery of retina below the inner limiting membrane embedded in the nerve fiber layer below the vitreous humor in the ganglion cell layer (FIGS. 3, 4, 5, and 6). They play a role in the transfer of therapeutic agents including chlorin e6 administered systemically (IV, subcutaneous) and orally. It is made of a tight junction of endothelial cells surrounded by basement membrane, pericytes, astrocytes, and Müller cells end feet and is akin to BBB of the CNS blood vessel. It does not play any significant role in transfer of Insulin, and chlorin e6 and other therapeutic agents from conjunctival sac to retinal photoreceptors, the site of pathology in night blindness and night vision.

2. The outer blood-retinal barrier is different from the inner blood retinal barrier. It formed by multiple posterior, long, and anterior ciliary arteries, not one central artery of the retina as seen in inner retinal blood barrier that is akin to blood brain barrier (BBB). They do have tight junction between the endothelial cells. Then, they give rise to myriads of fenestrated capillaries, which leak many components of the blood into the space surrounding them into choroid, on the external surface of the Bruch's membrane, RPE and from here; these leaked nutraceuticals permeate to the outer segments of the photoreceptors to maintain their structure and function (FIGS. 3, 4, 5, and 6). Once these Insulin, and chlorin e6 therapeutic agents and nutritional components leave the fenestrated BV of the inner layers of the choroid, they pass through the Bruch's membrane (acellular) and then tightly connected pigment epithelium to reach the photoreceptors outer segments (FIGS. 1-6). The one cell layer thick pigment epithelium with tight junctions plays a role in formation of outer blood-retinal barrier. The outer retinal-blood barrier delivers the Insulin and chlorin e6 therapeutic agents and when administered through these two routes, but does not allow any blood cellular constituents from the blood. It is the high vascular system of the choroid (uveal system), that transports therapeutic agents from the conjunctival sac to photoreceptors. First, the Insulin, and chlorin e6 transported through the transconjunctival, the scleral and choirdal blood vessels, through the multiple posterior, long, and anterior ciliary vessels as shown in the FIGS. 3, 4 and 5.

Some of the therapeutic agents transported through the posterior short ciliary, anterior ciliary and long ciliary arteries when parenteral or oral routes selected to deliver the therapeutic agents systemically. From the conjunctional sac, the therapeutic agents can migrate to retro bulbar space, and enter posterior short ciliary, anterior ciliary and long ciliary arteries and reach the choroidal circulation and delivered to the photoreceptors.

Both the internal and external retinal blood barriers associated blood vessels derived from the ophthalmic branch of the internal carotid artery. It is the rich vascular plexus of the uvea (choroid) and conjunctiva sac (FIGS. 3-5), plays a major role in our method of delivery of Insulin, chlorin e6 and adjuvant therapeutic agent's transport. We utilize this rich vascular supply of the sclera and choroid in the present invention to treat night blindness and enhance the night vision by delivering Insulin with chlorin e6 therapeutic agents to the photoreceptors, the site of visual pathology.

The therapeutic agents such as Insulin, chlorin e6, ketamine, IGF-1, and monoclonal antibodies deposited in the conjunctival sac to treat night blindness and to improve night vision do not reach directly into the inner blood-retinal barrier but passes through the outer retinal blood retinal barrier to reach the outer segment of the photoreceptors (FIGS. 1-6). That is why the delivery of therapeutic agents through the conjunctival sac is important and very effective compared to the systemic administration. This is specially the case for treating retinal and other ocupopathies which contribute to the night blindness and decreased night vision.

The therapeutic agents from the conjunctional sac (FIGS. 1, 2) have to travel no more than 3-10 millimeters to reach the site of pathology and need. When compared to hundreds of centimeters and meters of blood vessel the insulin, and chlorin e6 as well as other therapeutic agents have to travel to reach the central artery of the retina when administered orally or parenterally. Further, there is no second pass through the liver as it happens with oral intake of therapeutic agents. Due to this fact, they do undergo biochemical changes and diluted with 5000 ml (5 liters) of circulating blood reducing the concentration in the retina where needed the most. That is why various known Insulin and chlorin e6, as well as other pharmaceutical, biochemical, nurticeuticals, and biological agents or compounds have hard time reaching the outer segment of photoreceptors where they are needed to treat the night blindness and to improve night vision and alleviate oculopathy when administered IV, IM, subcutaneous and oral routes. The outer segments of the photoreceptors are ideally located right on the retinal pigment epithelium, directly in contact with inner cell membrane of the pigment epithelial cells, hence facilitates rapid transfer of insulin, and chlorin e6 and other adjuvant therapeutic agents for the treatment of night blindness and to improve night vision through the conjunctival sac delivery.

The therapeutic dose administered directly to the conjunctional sac is small (10% or less) compared to the oral and parenteral administration, which have adverse effect all over the body besides eyes. Further, by the time they reach the eyes, the therapeutic agents modified and altered in liver and other organs resulting in its reduced potency. Because the 85% of the blood from the choroid reaches the retina, hence 85% of the therapeutic agents from the conjunctional sac delivered to the photoreceptors of the retina compared to the systemic delivery.

Ketamine as NMDA Receptor Blocker, Protects Photoreceptors and Prevents Further Damage Due to Glutamate Mediated Excitotoxicity, Thus Maintain Proper Retinal Function, Prevent the Development of Night Blindness, Enhance the Night Vision, and Cure or Curtail the Underlying Disease Process.

Advantage of the present invention is that the with ketamine with insulin provides a therapeutic agent to treat retinitis pigmentosa, age related macular degeneration, diabetic retinopathy, likewise eye disease by blocking NMDA receptors, and thus block the excitotoxicity by extracellular glutamate released by damaged photoreceptors and RPE, retinal complex. There are multiple other therapeutic agents act as NMDA receptor blockers such as Remacemide, Memantine, Riluzole, Lamotrigine, Budipine, Gabapentin etc. We had the most experience and safety margins with ketamine compared to many others. It is important to note that the ketamine easily passes through the external and internal blood retinal barrier to reach retina, block the glutamate excitotoxicity, and prevent further advancement of the diseases in the photoreceptors. Our studies on hiccup showed that ketamine easily passes through the blood brain barrier (BBB) to reach the deep-seated neuronal masses in the area postrema of the fourth ventricle in the medulla oblongata and their connection (Shantha T R: Ketamine for the treatment of hiccup during and following anesthesia. A preliminary report. Anesthesia and Analgesia 52:822-824, 1973). In the same manner, it also reaches the photoreceptors afflicted in the above-described retinal photoreceptors afflictions through the conjunctival, choroidal vasculature. Ketamine is one of most important NMDA blocker, thus prevent the excitotoxicity effects of glutamate on the retina particularly on the photoreceptors. The micro doses of ketamine we use in the ophthalmic drops have no hallucinogenic or other ill effect. It is one of the ideal ophthalmic therapeutic agents for treatment of various retinal diseases including AMD, and retinitis pigmentosa; that contribute to night blindness and decreased night vision and other vision related symptoms. Pharmacologically, ketamine classified as an NMDA antagonist. The present inventor used this in thousands of case as dissociative anesthesia neuropathic pain, depression, and experiment show that it inhibits the rabies virus multiplication (U.S. Patent Application Publication Number: 2011/0020279 Al). The invention described herein incorporates ketamine in the ophthalmic drops delivered to the conjunctional sac. It is important to note that ketamine has mild local anesthetic effect and thus prevents the stinging-burning experienced after conjunctional sac instillation. Ketamine being a NMDA blocker, prevents excitotoxicity of glutamate on photoreceptors, and preserves the remaining photoreceptors intact to perceive vision in low light levels and in the dark.

Chlorophyll Derivatives and Chlorin e6 for Improving Night Vision and Treat Night Blindness

Water-soluble derivatives of chlorophyll first introduced as potential drugs by E. Snyder (USA) in 1942. The other step was that oral and intravenous administration of chlorin mixtures, mainly containing chlorin e6 are favored due to its low toxicity. Their usage has resulted in hypotensive, antisclerotic, spasmolytic; anesthetic, anti-rheumatoid outcome resulted in their usage for prevention and treatment of cardiovascular diseases, rheumatoid poly arthritis, and atherosclerosis and in photodynamic therapy (PDT) to treat cancers. The chlorophyll became part of the toothpaste in fifties and sixties and extensively used without ill effects. This is an indication that it is safe to use without adverse effects. So it can be used in the treatment of night blindness and improve night vision. Such a chlorophyll derivative is named chlorin e6 (synonyms: 2S-trans)-18-Carboxy-20-(carboxymethyl)-13-ethyl-2,3-dihydro-3,7,12,17-tetramethyl-8-vinyl-21H,23H-porphine-2-propionic acid).

Studies by Washington, et al., (IBID) showed that the water-soluble chlorophyll derivative chlorin e6 penetrate the mouse blood-retinal barrier and localize in mammalian photoreceptor cells that contain the visual pigments. They injected intravenously a solution of chlorin e6 (2.00 mg per kilogram body weight) which has to pass through the outer and inner blood retinal barrier. It is the outer retina barrier, which allows the chlorin e6 and such therapeutic agents to reach the outer segment of the photoreceptors especially rods to enhance the night vision and ameliorated night blindness. The chlorin e6 and other therapeutic agents from the central artery of the retina do not pass to the outer segment of the photoreceptors, but they do reach the bipolar and ganglion cell neuron. The above experiments showed that one hour after administration, the eyes removed and examined by fluorescence spectroscopy, showed a strong emission band of red florescence centered at 675 nm observed. This is due to uptake of the chlorophyll derivative chlorin e6 by the retina, mainly by the photoreceptors. In the controls eyecups of non-injected mice, this 675 nm band not observed. These findings indicate that the intravenously injected chlorophyll derivative chlorin e6 reaches the retina by passing through the outer blood-retinal barriers, and was deposited or taken by the photoreceptors outer segments from the rich choroidal blood vessels. These deposits remained in the photoreceptors for three hours post administration. These observations are in concurrence with similar experiments in rabbit models (R. Haimovici, et al. IBID). They also showed that the derivatives of chlorophyll act as visual pigments initiating the transformation of light into an electrical signal and thus change the primary event in vision to initial activation of a chlorophyll derivative. They also found that the electroretinographic amplitudes recorded in response to red and blue light were two-fold greater in mice administered chlorin e6, which is due to accumulated chlorophyll derivative in photoreceptor outer segments. Further, they also found that in the presence of chlorin e6, an almost two-fold increase in neural impulse generation in the retina in response to red and blue light, but not amber light. The stimulus wavelengths generating the increased visual response correspond to the absorption spectrum of chlorin e6. Since systemically injected chlorin e6 also localized in the retina, it is concluded that the increase in visual sensitivity is the result of light absorption by chlorin e6. The above data along with past research shows that derivatives of chlorophyll can act as visual pigments initiating the transformation of light into an electrical signal. In doing so, the primary event in vision changed from initial activation of the protein opsin to initial activation of a chlorophyll derivative. This mechanism shows to enhance vision in a mouse model. Our invention studies show that chlorin e6 with insulin enhance the night vision in humans, also. We prescribed Chlorella, which is also rich in chlorophylls with insulin for night blindness and decreased night vision with improvement in night vision. Other derivatives of chlorophyll also used when and if available with safety tract record as chlorin e6. As this chlorin e6, combined with insulin, absorbed by the retina particularly retinal rods, incorporated to the rods pigments. The insulin exerts its effect by acting as endocrine, intracrine, and paracrine modes by augmenting and amplifying the effects on the activity of these therapeutic agents through intracrine activity many. It also augments the uptake from extracellular space (Paracrine) and enhances its activity of chlorin e6 many times once within (intracrine) the photoreceptors.

This chlorin e6 extensively used as food colorant, non-toxic and easily available. We selected to use preparation of chlorophyll derivative chlorin e6 buffered to attain 6.4-7.4 pH (with NaHCO3). Each ml of this preparation contained 10, or 15 or 40 IU of insulin. Short and medium acting insulin used in our compounding ophthalmic drops. It is combined with 2 mg, 4 mg, or 5 mg or more per ml of chlorin e6 in combination or separately.

Insulin Role in the Uptake, Distribution; Augmentation—Amplification Effects of Therapeutic, Pharmaceutical, Biochemical and Biological Agents or Compounds Such as Chlorin e6, Ketamine and Monoclonal Antibodies on Photoreceptor Cells to Improve Night Vision, Treat Night Blindness and Associated Age Related Macular Degeneration (AMD), Diabetic Retinopathy, and Retinitis Pigmentosa Described Herein.

A variety of carriers, adjuvant agents, absorption enhancers, and facilitators, assist to get entry into the cell, potentiators of therapeutic action (augmentation/amplification effects), cell metabolic activity enhancers, cell multiplication enhancers, and other methods have been used. They administered to enhance the absorption and to potentiate the effect of therapeutic, pharmaceutical, biochemical, and biological agents or compounds within the afflicted cell, administered to the patients for improving the physiological function, and the treatment of diseases. Such an endocrine, paracrine, and intracrine biological agent is Insulin.

Insulin benefits the post ischemic myocardium by stimulating pyruvate dehydrogenase activity, which this activity in turn stimulates aerobic metabolism on cardiac and other tissue reperfusion with similar action on the photoreceptors. Insulin increases the glutathione synthesis by activating gamma-glutamyl-cysteine synthetase. Insulin decreases glutamate in the extracellular fluid that can damage the photoreceptors, by excitotoxicity by enhancing its uptake by the retinal cells. The insulin reduces both polymorphonuclear neutrophils adhesion due to ROS (reactive oxygen species-ROS-free radicals). Insulin affects the DNA, RNA, and protein synthesis; results in increased growth by mitosis (Osborne C K, et al. Hormone responsive human breast cancer in long-term tissue culture: effect of insulin. Proc Natl Acad. Sci. USA. 1976; 73: 4536-4540). Insulin increased redox status by increasing intracellular glutathione (GSH) content in oxidized cells. This reduced the ROS from the cells. It enhances the permeability of cell membranes to many therapeutic agents including chlorin e6. Besides glucose, and electrolytes, insulin helps facilitate the movement of the drugs and therapeutic agent molecules from extra cellular fluid (ECF) to intracellular fluid (ICF) meaning from outside the cells to inside the cells.

Insulin and similar biological agent IGFs have properties of tissue growth factors, and regulate growth and energy metabolism at the whole organism level farther away from the site of production and application in the conjunctival sac. This is the reason our invention with the use of Insulin with chlorin e6 topically not only has the local effect; they are absorbed and circulated farther away from the site of application and exert their therapeutic effects on the rods, cones, and in the retina to improve the night vision and teat night blindness.

Our and other clinical studies show that the Insulin and IGF-1 will exert endocrine, paracrine, intracrine effect, and enhance the absorption, and action of chlorin e6 inside the rods to enhance to night vision and alleviate night blindness. Once inside the cells, the insulin augments and amplifies the effects of chlorin e6 (intracrine effect) and any adjuvant agent proven and/or approved to treat night blindness and restoring their physiological function of the rods. The results show that increased production of GSH by insulin can reverse the effect of oxidation (ROS-oxidative free radical damage) by tyrosine kinase activation and phosphorylation.

In the research studies, the augment and amplifying effects of insulin been meticulously and conclusively demonstrated, that it activates and modifies metabolic pathways in MCF-7 human breast cancer cells by paracrine and intracrines effects. The studies showed that the insulin increases the cytotoxic effect (augment and amplify the effects) of methotrexate up to 10,000 (ten thousand) fold (Oliver Alabaster, et al., Metabolic Modification by Insulin Enhances Methotrexate Cytotoxicity in MCF-7 Human Breast Cancer Cells, Eur J Cancer Clinic; 1981, Vol 17, pp 1223-1228). Our studies for more than a decade support the findings of Alabaster (IBID), that the disease or the healthy cell's sensitivity to the therapeutic and biological agents augmented and amplified by the effects of insulin, as those used to enhance night vision and treat night blindness. This can be increased (augmentation/amplification effects) many times by using the method described in this invention using insulin with chlorin e6 to treat decreased night vision and night blindness. Insulin can independently stimulate retinal cells growth in eye structures including photoreceptor and RPE cells (Shantha T. R., Unknown Health Risks of Inhaled Insulin. Life Extension, September 2007 pages 74-79, Post publication comments in September 2008 issue of Life Extension, Pages 24. Shantha T. R and Jessica G. Inhalation Insulin, Oral and Nasal Insulin Sprays for Diabetics: Panacea or Evolving Future Health Disaster. Part I: Townsend Letter Journal: Issue #305, December 2008 pages: 94-98; Part II: Townsend Letter, January 2009, Issue #306, pages-106-110). The application of the combinations of insulin with chlorin e6 into conjunctival sac can deliver this chlorophyll and its derivative such as chlorin e6 to rods. Thus, the insulin can enhance the uptake of chlorin e6 and its function as night vision enhancer, and alleviate the night blindness and improve the night vision especially in the elderly.

Insulin improves cellular physiological function. In addition, insulin augments/amplifies the effects of therapeutic agents (Alabaster IBID) such as chlorin e6 when used in combination locally for treatment of night blindness and enhances the night vision. Hence, invention described herein, with local use of insulin alone or with other therapeutic agents such as chlorin e6 is very effective in treating night blindness and improving night vision and related afflictions of the retina. In this invention, the use of Insulin, chlorin e6; with or without ketamine, monoclonal antibodies, and IGF-1, based on meeting all the above-recited physiological, pharmacological, and therapeutic parameters.

It is important to note that the Insulin-like growth factor-1 (IGF-1) and insulin have similar three-dimensional structures and similar function and it is a single-chain polypeptide of 70 amino acids.

IGF-1 is a trophic factor circulates at high levels in the blood stream. It is a much more effective neurotrophic factor compared to insulin and has positive effect on the photoreceptors in repairing and maintaining functioning in the retina and the photoreceptors, especially in retinitis pigmentosa and dry age related macular degeneration. The insulin augments and amplifies the effects of IGF-1 in the photoreceptors. IGF-1 influence neuronal structure and functions throughout the life span. Studies have shown the effect of IGF-1 on the hair cell growth of the inner ear. The IGF-1 has the ability to preserve nerve cell function specially photoreceptors and promote nerve growth in experimental studies.

The retina is nothing but the extension of the brain; hence, the effect of therapeutic agents on the retinal photoreceptors is similar to the effects on the CNS neurons. Hence, the IGF-1 and insulin play an important role in maintaining proper integrity, growth, repair, and functioning of the eye and retina, in particular. Because of these properties, recombinant human IGF-1 is in clinical trials for the treatment of amyotrophic lateral sclerosis (ALS). The primary function of IGF-1 is to stimulate cell growth in every part of the body including retina. The body builders use 100 mcg to 400 mcg per shot without concern and ill effect for its anabolic effects. Its effect in the treatment of night blindness can have implication and can augment and amplify the effects of chlorin e6 in combination as ophthalmic drops similar to and with insulin.

Caution: Because of the angiogenesis effects IGF-1 has, we do not include this therapeutic biological agent in the compounded ophthalmic drops used to enhance night vision and treat night blindness associated with wet AMD, diabetic retinopathy, eye tumors, and other oculopathies associated with angiogenesis. Because, it has angiogenesis effect on the BV, its application can aggravate angiogenesis associated oculopathies, hence avoid its use in such conditions (Antoinette C Lambooij et al, Insulin-like Growth Factor-I and its Receptor in Neovascular Age-Related Macular Degeneration. Investigative Ophthalmology & Visual Science. May 2003, Vol. 44, 3, 2192-2198. Claudio Campa et. al. Inflammatory Mediators and Angiogenic Factors in Choroidal Neovascularization: Pathogenetic Interactions and Therapeutic Implications. Mediators of information, Volume 2010, Article ID 546826, 14 pages. Rita Rosenthal et al. Insulin like growth factor-I contributes to neovascularization in age related macular degeneration. Biochemical and Biophysical Research Communications 323 (2004) 1203-1208). Only chlorin e6 with or without insulin are used in such patients.

Oxidizing Agents to Facilitate the Delivery of Insulin Through the Conjunctional Mucosal Lining to the Choroid and Retina

As described herein, the pharmaceutically acceptable oxidizing agent facilitates the delivery of the bioactive agent through the mucous membrane. In general, the oxidizing agent can react with molecules present in the conjunctional sac mucosal membrane (and skin epidermal layers) that would adversely react with the bioactive agent. For example, reduced glutathione present in the skin inactivate bioactive agents by breaking crucial molecular bonds. Not wishing to be bound by theory, when delivering insulin either transmucosally or transdermally, reduced glutathione can inactivate insulin. Specifically, insulin has numerous disulfide bonds, which are crucial for its protein conformation, biological activity, and subsequent therapeutic effects. Reduced glutathione will inactivate insulin by reducing or breaking insulin's disulfide bonds. Once these disulfide bonds are broken, insulin becomes inactive due to lost protein conformation and biological activity. Thus, the administration of the oxidant or oxidizing agents using the method described herein prevents the inactivation of the bioactive agent insulin by reducing the effect of reduced glutathione. Specifically, applying an oxidant or a pharmaceutically oxidizing agent transmucosally will lower or prevent the effects reduced proteins and reduced biological molecules have on the bioactive agents. In this manner, the inactivation of bioactive agents via reduction or cleavage of crucial molecular bonds avoided. The selection and amount of the pharmaceutically acceptable oxidizing agent can vary depending upon the bioactive agent administered.

In one aspect, the oxidizing agent includes, but is not limited to, iodine, povidone-iodine, and any source of iodine or combinations of oxidants, silver protein, active oxygen, potassium permanganate, hydrogen peroxide, sulfonamides, dimethyl sulfoxide, or any combination thereof. These oxidizing agents may also act as absorption agents; that help to facilitate delivery of a therapeutic agent onto, into, and across a mucosal membrane of the conjunctional sac.

Interestingly, the conjunctional lining has very thins layer of strum corneum compared to the skin, palm of the hand, planter surface of the feet; hence hardly any glutathione to inactivate the insulin. We have used povidone iodine in our studies as an oxidizing agent of glutathione to neutralize the effect of reduced glutathione if any in the conjunctional sac. We use 0.1% to 0.05% povidone iodine (PVP-I) solution in normal saline in this invention. The dose can be increased or decreased. Insulin compounded PVP-1 solution with 40 IU of insulin per milliliter, delivered to the conjunctival sac as drops with insulin. 2.5% buffered PVP-I solution is already in use for prophylaxis of neonatal conjunctivitis (Ophthalmia neonatorum) which can lead to blindness, especially if it is caused by Neisseria gonorrhoeae, or Chlamydia trachomatis. PVP-I is suitable for this purpose because unlike other substances it is efficient also against fungi and viruses (including HIV and Herpes simplex). It has proved harmless to ocular structures in the newborn so also in adults.

Preparation of the Patients and Ophthalmic Drops to Treat Night Blindness and Enhance the Night Vision Using this Inventive Method of Using Insulin, with, Chlorin e6 and Adjuvant Therapeutic Agents Such as IGF-1, Ketamine and Monoclonal Antibodies

Before using described inventive methods and examples, a thorough examination of the affected patient's eye is in order. The examination of the eye may include 1. Acuity testing, 2. Biomicroscopy, 3. Intraocular pressure (IOP), 4. Ophthalmoscopy, 5. Color vision test, 6. most important test, an electroretinogram (ERG) to measure the function of the photoreceptors. If needed, additional tests could be done such as 7. Schimer's test, 8. Tear film breakup time (tBUT), 9. Test for Superficial punctate keratitis (SPK), 10. Fluorescein and Rose Bengal staining (RBS) of BV of the retina, as well as cornea, conjunctiva, and eyelids, 11, slit-lamp examination of the conjunctiva, cornea, anterior chamber, iris, and lens, 12. The Ocular Surface Disease Index (OSDI), 13. microscopic examination of the tear filament, 14. Maturation index (a Papanicolaous stained sample of conjunctival epithelium).15. Tear osmolality. In addition, a complete physical examination with blood test for thyroid, parathyroid, growth hormone, insulin, IGF-1, FSH, LH, cortisol, estradiol, and testosterone levels, electrolytes, blood cell count, cholesterol levels, ESR, are advised. The urine sample for pregnancy test if the patient is of childbearing age deemed necessary. In those who are nighttime workers, soldiers, hunters, and night drivers etc. without the history of retinal diseases and other oculopathies, most of the above test not needed and all they need is a simple routine eye examination by an ophthalmologist.

To instill ophthalmic insulin drops of this invention into the conjunctional sac as therapeutic agents, the patient will wash their hands with mild, antiseptic soap. The person or patient applying the drops must be careful not to touch the dropper tip to the eyelids (and any foreign objects) to avoid contamination if there is an eyelid infection. Tilt the head back, or lay down with head extended on a neck pillow; gaze upward and backwards; and pull down the lower eyelid to expose the conjunctival fornix. Place the dropper directly over the eye away from the cornea and instill the prescribed number of drops. Look downward and gently close your eye for 1 to 2 minutes. The patient should not rub the eye harshly. Do not rinse the dropper unless the patient or person knows the sterilization technique with hot water. If other therapeutic, pharmaceutical, biochemical, and biological agents or compounds are to be selected to treat the condition with this invention, wait 5 or more minutes before using other selected anti-night vision and night blindness therapeutic agents or the other variety of ophthalmic medicaments. It is important to instill medications regularly as prescribed to enhance the night vision and prevent or reduce night blindness.

Ophthalmic drops compounded as described below instilled at least 2 hours before working in the dark or night driving or before sunset and at bedtime. Consult your doctor and/or pharmacist if the systemic medications that you are currently taking are safe to use with the eye drops described and prescribed. There is no contraindication for the insulin eye drops, except the patients with hypoglycemia syndromes, and in some cases, external ocular tumors.

To minimize the absorption insulin into the bloodstream and to maximize the amount of drug absorbed by the eye, close your eye for one to five minutes after administering the insulin drops. Then, press your index finger gently against the inferior nasal corner of your eyelid to close the tear duct that drains into the nose (FIG. 7). This will prevent any adverse systemic effect due to nasal vascular uptake into the systemic circulation from the nasolacrimal duct drainage of the therapeutic agent insulin from the conjunctival sac. Eye drops may cause a mild, uncomfortable burning or light stinging sensation; this reaction should last for only a few seconds. The anti-night vision and night blindness drops take effect within 30-60 minutes after application depending upon the therapeutic agents used with the eye drops. We recommend the best time to use insulin eye drops with chlorin e6 is at about 5-6 PM before dark and at about 9.00 PM before sleep. This process repeated every 6-8-12 hourly for 4-7 days a week until the desirable results obtained. To improve night vision and the patients with night blindness with retinal afflictions can use insulin eye drops with chlorin e6 all their lives or intermittently, depending on the results and the need. The therapeutic agents are instilled using a sterile dropper (or bottle with medication equipped with a dropper nipple) into the conjunctival sac.

Preparation of Insulin, Chlorin e6, with Povidone Iodine Eye Drops for Use to Enhance Night Vision and Treat Night Blindness

Take 200 international units (IU) of rapid or intermediate or long acting insulin and dilute in 5 ml (40 IU/ml) of sterile saline or distilled water or other carriers and facilitators as described above. The pH adjusted to prevent the sting when the insulin instilled into the conjunctival sac. This preparation can contain nanograms (mini micrograms) of local anesthetics to prevent the stinging when the eye drops. In this preparation, each ml contains 40 units of insulin. In pharmacies, another name for a drop is ‘minim,’ (a drop would be 0.0616 milliliters). The drop standardized in the metric system to equal exactly 0.05 milliliters. The 20 drops equal one ml (1 cc) which each drop contains 2.0 IU of insulin. The concentration of the insulin content decreased or increased to 0.20, 0.30, 0.40, and 0.50 or up to 1 IU of insulin per drop. The insulin content increased per drop in the dilutent preparation as desired. To this preparation add povidone iodine to a concentration of 0.1% or 0.05% per mil as oxidizing antiseptic agent. The insulin content decreased by reducing the insulin units used for the preparation of the ophthalmic drops. Instill one to two drops or three drops to each eye lower lid fornix and/or everted upper eyelid (conjunctival sac) as a single agent. The applicant must apply pressure on the nasolacrimal duct as shown in the FIG. 7 to prevent drainage into the nasal cavity.

If other combinations of the anti night vision and night blindness therapeutic agents are to be used, first, use insulin drops, wait for 3-5 minutes and apply the other adjuvant therapeutic, pharmaceutical, biochemical, and biological agents or compounds. After this procedure, instill one more insulin drop further enhance the uptake of the other selected therapeutic agents to augment/amplify their effects at the cellular level. This step is optional. The dose used in our invention can be appropriately selected depending upon symptom, age, severity of the disease, dosage form, and existing health conditions. The pH can be within a range acceptable to ophthalmic preparations which the pH preferably is within a range from 4-6-7 most preferably 7.4. The pH adjusted with use of sodium bicarbonate (NaHCO₃).

There are various forms of insulin used to treat diabetes. Insulin products categorized according to their putative action as rapid, short, intermediate, and long acting insulin. We have used rapid acting, short acting, and long acting protamine zinc insulin in our studies. Protamine Zinc Insulin is long acting insulin contains Zinc. Zinc is an antioxidant, hence this form of insulin even more effective in reducing the effect of ROS. Because of its zinc content, this form of insulin more often in compounding of our ophthalmic drops.

There is a possibility of developing hypoglycemia when the insulin used as indicated by signs and symptoms such as rapid heartbeat, sweating, dizziness, confusion, unexplained fatigue, shakiness, hunger, feeling hot, difficulty in thinking, confusion. Such patients should be treated with oral ingestion of a fast-acting carbohydrate such as glucose tablets, fruit juice, fruit bowl, chocolate bar, regular Coca-Cola, sugary drinks or eat plain sugar followed with a drink of water or IV administration of 25% glucose, if the reaction is severe.

Insulin compounded as a liquid ophthalmic isotonic solution containing cyclosporin, or other antiautoimmune therapy agents, adjuvant therapeutic agents, or vitamins, and one or more one buffering agents, said buffering agents producing a pH in said composition similar to mammalian eye fluids.

The data supports the other therapeutic agents, used after insulin where the agents are prepared in 2-5% solutions of glucose. The glucose acts as a carrier of the therapeutic agents after pretreatment with insulin. I have named this method as local Insulin Potentiation Therapy (LIPT). The insulin pharmaceutical eye drop preparation of this invention may contain 0.25%-0.5%-1%-2% or more glucose. There are several mechanisms in which glucose and insulin protect the damaged cells and the insulin restores them normal function. Glucose with the help of insulin esterifies intracellular free fatty acids which the fatty acids, decreases their toxic products and oxygen free radicals.

The pharmaceutical eye drop preparation of our invention may contain antibacterial components, buffering ingredients, tonicity imparting agents, absorption enhancers, and preservative agent such as EDTA. They may also compounded with non-noxious pharmaceutical carrier, non-toxic emulsifying, nonionic surfactant, preserving, bodying agents, wetting agents, lubricant such as cellulose derivatives (carboxymethyl cellulose) including, a conventional phosphate buffer vehicle systems, isotonic boric acid vehicles, isotonic sodium chloride vehicles and the like

### Example 1

In this ophthalmic preparation, take 5 ml of normal saline, which contains 0.05% povidone iodine premixed. To each ml add:

a) short acting insulin 40 IU,

### b) Chlorin e6, 20 mg,

c) EDTA 30 mcg as preservative,
d) Prepare in a 5 or 10 ml sterile bottle with an eyedropper or plastic squeeze dropper. The dispenser is pre sterilized in boiling water or in a pressure sterilizer before mixing the above contents.
e) Mix them well in pharmaceutical shaker for 15 minutes under strict aseptic conditions and store in a sterile clean cool refrigerator until used.
The composition can be dispensed as liquid drops, ointment or as gel deposited under the eyelids. These therapeutic agents can also be delivered through the conjunctival sac by inserting a slow releasing non-reacting, self absorbing gel-patch containing insulin and chlorin e6 and other adjuvant therapeutic agents, inserted into the lower fornix of the conjunctival sac under the lower lids for prolonged slow release of therapeutic agents to be instilled specially before going to sleep.
Case report: This is a 65-year-old male, who went deer hunting in the wee hours of the morning, in the cover of the darkness, when the sun has not yet risen during deer hunting season. He had difficulty of vision at dusk as he aged compared to when he used to hunt without difficulty when young. He used prescriptions glasses and had cataract surgery. His poor night vision continued. He used the ophthalmic preparation as described above before going to bed and before going hunting. His vision improved according to subjective report after a week of use. He uses the eye drops routinely at night before going to bed and before sunset. His night vision also improved and does not bump into objects as it used to happen in the home due to poor night vision. He uses these drops routinely even during non-hunting season.

### Example 2

Instead of premixing as described in the example 1, we have dispensed each therapeutic agents in separate dispensers with following instructions:

a) Eye drops contain chlorin e6 10 mg per/ml, prop 2-3 drops per eye. Each drop of ophthalmic drop will contain 0.50 mg of chlorin e6,
b) Wait for 5 minutes,
c) Then, instill 2-3 drops of insulin (one ml containing 40 units of insulin in 0.05% povidone iodine),
d) Then, rest lying down for 5-10 minutes and then resume activities.
e) It is important to use these ophthalmic preparations instilled to conjunctional sac around 5-6.00 PM, then before going to bed.
f) During daytime, use it as the sun goes down, or eight hourly in night blindness associated with oculopathies.
g) Daytime application avoided in young with normal vision and without eye diseases. Such person engaged in nighttime avocations; use these ophthalmic preparations in the evening and at bedtime and at 3.00 AM at work or driving to enhance the night vision.
If there are retinal photoreceptors changes as seen in age related macular degeneration (dry AMD) and retinitis pigmentosa, add ketamine 50 mcg per ml in the preparation, and use it before the insulin drops instilled into the conjunctival sac.

### Example 3

a) Prepare eye drops as described in example 2,
b) Drop 2-3 drops per eye before going to bed, and at 6 PM.
c) Wait for 15 minutes,
d) Then, rest lying down for 10 minutes and then resume night work,
e) It is important to use this preparation before going to bed, and next day around 6:00 PM and eight hourly.
f) If the person is a night shift worker, patient instructed to carry it in pocket, and apply every eight hourly. For such patients, only chlorin e6 drops prepared and used with or without insulin.
Case report: This is a nighttime factory worker aged 55. He has gradually developed decreased night vision and night blindness. He has cataract changes in the eye, not advanced enough to perform cataract surgery. We prescribed the above regimen. Ask him to use every eight hourly after sundown, and eight hourly at daytime. His vision improved and was at ease driving at night times to work and at work.

### Example 4

Case report: This is a 58 years old male patient diagnosed with retinitis pigmentosa with increasing symptoms as sun went down and at night. This was an early case of retinitis pigmentosa. He kept bumping into objects at work and home. He had difficulty in peripheral vision (tunnel vision). We prepared the following ophthalmic preparation and dispensed it.

a) Prepare eye drops containing chlorin e6, 10 mg per/ml, and IGF-1, 100 mcg per/ml. Add to this preparation 60 IU of insulin in 0.05% povidone iodine solution.
b) The above therapeutic agents, we added Ketamine, 50 mcg/ml in saline,
c) The above compounds compounded with EDTA as preservative. The pH adjusted to 7.00 by using NaHCO₃ as described above.
d) Drop 2-3 drops per eye, at 6 PM and before going to bed.
e) Then, rest lying down for 15 minutes with head extended and then resume nighttime activities such as driving or working.
f) He was instructed to use the eye drops every 8 hourly during daytime.
After 3 months of use, his decreased night vision (night blindness) improved. The tunnel vision also decreased and peripheral vision improved. His vision continues to improve without any further deterioration. The ketamine in these ophthalmic drops prevents the excitotoxicity of glutamate released by degenerating photoreceptors, Müller cells, and RPE and enhance the glutathione antioxidant to neutralize the ROS damage. The IGF-1 is a neurotrophic factor, and in the present preparation helps to bring back the degenerating, dysfunctional rods and preventing their further apoptosis. Our study showed that the improvement in the vision changes are more dramatic with ketamine-included drops compared to those without it. The patient put on lutein and vitamin A rich diet. At nighttime, the patient provided with eye cooling mask to maintain temperature of 22-24° C. worn during sleep. This cooling of the eyes reduced the metabolism in photoreceptors that results in reducing the generation of the free radical. This is due to lowered metabolism of genetically defective photoreceptors, thus reduce the ROS retinal damage in retinitis pigmentosa.

### Example 5

The night workers such as soldiers, police, drivers, factory workers without retinal diseases and other oculopathies need not include insulin in the ophthalmic preparations.

a) Prepare Eye drops contain chlorin e6, 10 mg per/ml, with 0.5% dimethyl sulfoxide (DMSO) in normal saline.
b) Drop 2-3 drops per eye. Each drop of ophthalmic drop will contain 0.50 mg of chlorin e6.
c) These drops used at 5.00 PM, at 8.00 PM and before going to sleep
d) Then resume night avocations.
Case reports: We have provided these ophthalmic drops to many security men, nighttime factory workers, and police, who reported reduced night blindness increased vision at night time.

### Example 6

Case report: This is a 66 years old man diagnosed with age related macular degeneration (dry type) with night blindness. Recommend to include in the diet, green leafy vegetables such as Kale, and Turnip green rich in lutein. He was put on oral extra lutein intake. He was on regimen of fish in his meal, three times a week with oral statin drugs to lower the cholesterol at bedtime. Red meat and dairy products were restricted and/or not allowed in the diet. He was also put on nightly intake of statins to reduce cholesterol levels. Lutein ophthalmic drops with chlorin e6 were prepared and provided as described.

a) It is prepared in a separate ophthalmic solution dispenser. Take 10 mg of lutein and add to 10 ml or normal saline.
b) Each drop of the ophthalmic preparation will contain 50 mcg of Lutein.
c) It is prepared as described in the example 1.
d) Instill this ophthalmic preparation before application of Insulin, chlorin e6 drops as described in the example 1.
The patient reported improvement in central vision and there was overall improvement in his night vision. The symptoms of AMD reduced and this oculopathy did not progress further.

### Example 7

Monoclonal antibodies are laboratory created substances that the antibodies can locate and bind to TNF, cytokines, and autoantibodies involved in the production of ROS and retinitis pigmentosa as well as other autoimmune diseases. Follow the instruction as described in the above EXAMPLE 1.

a. Apply ophthalmic solution of Monoclonal antibodies. We formulate Etanercept (Embrel) using 400 μg per ml of ophthalmic solution, which results in 20 μg per drop instilled.
b. Add to the above drops, 50-mcg ketamine with Etanercept.
c. It is important to use these ophthalmic preparations before going to bed, and next day every eight hourly. The patient's night blindness and decreased night vision improved. The tunnel vision associated with retinitis pigmentosa reduced and did not progress further.

Numerous modifications, alternative arrangements of steps explained, examples given herein may be devised by those skilled in the art without departing from the spirit and the scope of the present invention. The appended claims intended to cover such modifications and arrangements. Thus, the present invention has been described above with particularity and detail in connection. This is presently deemed to be the most practical and preferred embodiments of the invention. The invention will be apparent to those of ordinary skill in the art that numerous modifications, including, but not limited to, variations in size, materials, shape, form function, and manner of procedure, assembly, and the use may be made. The preferred embodiment of the present invention has been described. The invention should be understood that various changes, adaptations, and modifications may be made thereto. It should be understood, therefore, that the invention is not limited to details of the illustrated invention. This method can be used to treat various oculopathies bedsides being used for treatment night blindness and improving the vision in the dark. The preferred embodiments of the present invention have been described. This should be understood, therefore, that the invention is not limited to details of the illustrated invention examples. Other features and advantages of the present invention will become apparent to one with skill in the art upon examination of the descriptions and drawings. It is intended that all such additional features and advantages be included herein within the scope of the present invention, as defined by the claims.

What is claimed is:

1. A method for treating night blindness and improving night vision, comprising the steps of:
placing a patient's head in a supine position with head extended allowing access to an afflicted vertebrates eye's conjunctival sac;
instilling a preparation topically consisting of insulin and chlorin e6, and their pharmaceutically acceptable salts thereof to said afflicted vertebrates eye's conjunctival sac, wherein each ml of said insulin and chlorin e6 contains 40 units of insulin, and 20 mg of Chlorin e6 in a normal saline solution also containing 0.05% povidone iodine and buffered with EDTA as preservative, and pH adjusted to be generally at 7.4 using a standard liquid dropper or a pre-sealed dropper plastic squeeze bottle;
waiting five minutes wherein said preparation is absorbed therein; and
repeating said previous steps at bedtime and every eight hours during waking periods.

2. The method for treating night blindness and improving night vision of claim 1, wherein said vertebrate is a human.

3. The method for treating night blindness and improving night vision of claim 1, wherein said vertebrate is a mammal.
